SEQUENCE LISTING (94 sequences)

SEQ ID NO: 1 - protein, 47 aa, Arabidopsis thaliana
WVRCDDCFKWRRIPASVVGSIDESSRWICMNNSDKRFADCSKSQEMS

SEQ ID NO: 2 - protein, 47 aa, Arabidopsis thaliana
VQCEKCMKWRKIDTQDEYEDIRSRVQEDPFFCKTKEGVSCEDVGDLN

SEQ ID NO: 3 - protein, 52 aa, Arabidopsis thaliana
TVQCASCFKWRLMPSMQKYEEIREQLLENPFFCDTAREWKPDISCDVPADIY

SEQ ID NO: 4 - protein, 43 aa, Arabidopsis thaliana
AAQCWKCLKVRSIESQEDYEEIRSKTLEKFFECKRCEEPGDMV

SEQ ID NO: 5 - protein, 47 aa, Arabidopsis thaliana
AAQCDNCHKWRVIDSQEEYEDIRSKMLEDPFNCQKKQGMSCEEPADI

SEQ ID NO: 6 - protein, 42 aa, Arabidopsis thaliana
WATCDDCSKWRRLPVDALLSFKWTCIDNVWDVSRCSCSAPEE

SEQ ID NO: 7 - protein, 44 aa, Arabidopsis thaliana
WVQCDACGKWRQLPVDILLPPKWSCSDNLLDPGRSSCSAPDELS

SEQ ID NO: 8 - protein, 45 aa, Arabidopsis thaliana
KHCDKPGTVEKMLICDECEEAYHTRCCGVQMKDVAEIDEWLCPSC

SEQ ID NO: 9 - protein, 45 aa, Arabidopsis thaliana
WMQCDSCSKWRRIIDEGVSVTGSAWFCSNNNDPAYQSCNDPEELW

SEQ ID NO: 10 - protein, 47 aa, Arabidopsis thaliana
WVACDKCGKWRLLPFGVFPEDLPEKWMCTMLNWLPGVNYCNVPEDET

SEQ ID NO: 11 - protein, 47 aa, Arabidopsis thaliana
WAQCESCEKWRLLPYDLNTEKLPDKWLCSMQTWLPGMNHCGVSEEET

SEQ ID NO: 12 - protein, 48 aa, Homo sapiens
WVQCSFPNCGKWRRLCGNIDPSVLPDNWSCDQNTDVQYNRCDIPEETW

SEQ ID NO: 13 - protein, 48 aa, Mus musculus
WVQCSSPKCEKWRQLRGNIDPSVLPDDWSCDQNPDPNYNRCDIPEESW

SEQ ID NO: 14 - protein, 49 aa, Homo sapiens
WVQCENENCLKWRLLSSEDSAKVDHDEPWYCFMNTDSRYNNCSISEEDF

SEQ ID NO: 15 - protein, 49 aa, Mus musculus
WVQCENESCLKWRLLSPAAAAAVNPSEPWYCFMNTDPSYSSCSVSEEDF

SEQ ID NO: 16 - protein, 46 aa, Homo sapiens
QCDLCLKWRVLPSSTNYQEKEFFDIWICANNPNRLENSCHQVECLP

SEQ ID NO: 17 - protein, 46 aa, Mus musculus
QCDLCLKWRVLPSSSNYQEKGLPDLWICASNPNNLENSCNQIERLP

SEQ ID NO: 18 - protein, 48 aa, Homo sapiens
TIQCDLCLKWRTLPFQLSSVEKDYPDTWVCSMNPDPEQDRCEASEQKQ

SEQ ID NO: 19 - protein, 43 aa, Mus musculus
TIQCDLCLKWRTLPFQLSAVEEGYPINWVCSMNPDPEQDQCEA

SEQ ID NO: 20 - protein, 37 aa, Homo sapiens
WVQCDACLKWRKLPDGMDQLPEKWYCSNNPDPQFRNC

SEQ ID NO: 21 - protein, 36 aa, Mus musculus
VQCDACLKWRKLPDGIDQLPEKWYCSNNPDPQFRNC

SEQ ID NO: 22 - protein, 59 aa, Arabidopsis thaliana
QCSAVNWLQCREEDTNGVICGKWRRAPRSEVQTKDWECFCCFSWDPSRADCAVPQELET

SEQ ID NO: 23 - protein, 59 aa, Arabidopsis thaliana; identical to SEQ ID NO: 22

SEQ ID NO: 24 - protein, 56 aa, Arabidopsis thaliana
QCSAVNWLQCREEDTNGVICGKWRRAPRSEVQTKDWECFCCFSWDPSRADCAVPQK

SEQ ID NO: 25 - protein, 59 aa, Arabidopsis lyrata
QCSAVNWLQCREEDSNGDICGKWRRAPRSEVQTKDWECFCCVFWDPSRADCAVPQELET

SEQ ID NO: 26 - protein, 60 aa, Zea mays
SIGNWIQCRETLNPGDSDKQVVCGKWRRAPLYVVQSDNWDCFCCLLWDPVHADCAVPQEL

SEQ ID NO: 27 - protein, 60 aa, Ricinis communis
SLSNWLQCQEVLYDETGEPIEGTKCRKWRRAPLSEVQTDEWDCSCSVTWDPFHSDCAVPQ

SEQ ID NO: 28 - protein, 60 aa, Oryza sativa
SIGNWIQCREILSEGDSDKPVVCGKWRRAPLFVVQSDDWDCSCCLPWDPAHADCAVPQEL

SEQ ID NO: 29 - protein, 8 aa, Artificial Sequence
  Synthetic
DYKDDDDK

SEQ ID NO: 30 - protein, 16 aa, Artificial Sequence
  Synthetic
ARTKQTARKSTGGKAY

SEQ ID NO: 31 - DNA, 25 bp, Artificial Sequence
  Synthetic
ataaccgtttcaaattctctctctc

SEQ ID NO: 32 - DNA, 20 bp, Artificial Sequence
  Synthetic
tgcaaatcgttctctccttg

SEQ ID NO: 33 - DNA, 25 bp, Artificial Sequence
  Synthetic
ccgcaagatcaagacgaaggatagc

SEQ ID NO: 34 - DNA, 25 bp, Artificial Sequence
  Synthetic
ccctgaggagcacccagttctactc

SEQ ID NO: 35 - DNA, 25 bp, Artificial Sequence
  Synthetic
gtttcggtttcacccgactgatgag

SEQ ID NO: 36 - DNA, 23 bp, Artificial Sequence
  Synthetic
tcgccttgcctaaaccattcaaa

SEQ ID NO: 37 - DNA, 18 bp, Artificial Sequence
  Synthetic
atatgcctccccctctgc

SEQ ID NO: 38 - DNA, 24 bp, Artificial Sequence
  Synthetic
ggatcatcttcttgatacagacca

SEQ ID NO: 39 - DNA, 22 bp, Artificial Sequence
  Synthetic
acccacgttttctctctttctc

SEQ ID NO: 40 - DNA, 22 bp, Artificial Sequence
  Synthetic
ggccattttttgtgatgcttaa

SEQ ID NO: 41 - DNA, 20 bp, Artificial Sequence
  Synthetic
cagggtgtcaagctctcgat

SEQ ID NO: 42 - DNA, 20 bp, Artificial Sequence
  Synthetic
ctgtgatggctgaggtttca

SEQ ID NO: 43 - DNA, 22 bp, Artificial Sequence
  Synthetic
cccacaacacttgtcttcatgt

SEQ ID NO: 44 - DNA, 20 bp, Artificial Sequence
  Synthetic
tgaccaacatggccaaacta

SEQ ID NO: 45 - DNA, 22 bp, Artificial Sequence
  Synthetic
cggttctgtgtttgtttgtgtt

SEQ ID NO: 46 - DNA, 21 bp, Artificial Sequence
  Synthetic
tcgtgaatgacatgcaatttt

SEQ ID NO: 47 - DNA, 20 bp, Artificial Sequence
  Synthetic
tccatcgtctccttccagac

SEQ ID NO: 48 - DNA, 20 bp, Artificial Sequence
  Synthetic
cttggcctcagtcacacctt

SEQ ID NO: 49 - DNA, 31 bp, Artificial Sequence
  Synthetic
tagggttcttagttgatcttgtattgagctc

SEQ ID NO: 50 - DNA, 28 bp, Artificial Sequence
  Synthetic
tttgctctcaaactctcaattgaagttt

SEQ ID NO: 51 - DNA, 20 bp, Artificial Sequence
  Synthetic
agtgacttgtcgactgcttt

SEQ ID NO: 52 - DNA, 20 bp, Artificial Sequence
  Synthetic
cccttatcggagatttgaag

SEQ ID NO: 53 - DNA, 20 bp, Artificial Sequence
  Synthetic
tctccagcatttccaagatc

SEQ ID NO: 54 - DNA, 20 bp, Artificial Sequence
  Synthetic
acggacagagcagtctcaag

SEQ ID NO: 55 - DNA, 22 bp, Artificial Sequence
  Synthetic
caaaactggcggggctaaggag

SEQ ID NO: 56 - DNA, 25 bp, Artificial Sequence
  Synthetic
ctccactgcaatcttcgaccatcta

SEQ ID NO: 57 - DNA, 22 bp, Artificial Sequence
  Synthetic
caccgtattcaatggttttagc

SEQ ID NO: 58 - DNA, 23 bp, Artificial Sequence
  Synthetic
tgctacgtagatgaaacgagttt

SEQ ID NO: 59 - DNA, 23 bp, Artificial Sequence
  Synthetic
ttgctttaaagcgcgacgaatac

SEQ ID NO: 60 - DNA, 23 bp, Artificial Sequence
  Synthetic
gtattcgtcgcgctttaaagcaa

SEQ ID NO: 61 - DNA, 23 bp, Artificial Sequence
  Synthetic
gagctctagagcgatctgtatga

SEQ ID NO: 62 - DNA, 23 bp, Artificial Sequence
  Synthetic
tcatacagatcgctctagagctc

SEQ ID NO: 63 - DNA, 23 bp, Artificial Sequence
  Synthetic
ctcaaaatctgcagagatgtcaa

SEQ ID NO: 64 - DNA, 23 bp, Artificial Sequence
  Synthetic
ttgacatctctgcagattttgag

SEQ ID NO: 65 - DNA, 23 bp, Artificial Sequence
  Synthetic
aaaatctcaagcgatgtcaaatg

SEQ ID NO: 66 - DNA, 23 bp, Artificial Sequence
  Synthetic
catttgacatcgcttgagatttt

SEQ ID NO: 67 - DNA, 69 bp, Artificial Sequence
  Synthetic
cggaattcatggttgtggatgttactattgaagatagctattccacagagagtgccgcggttcgatgtg

SEQ ID NO: 68 - DNA, 23 bp, Artificial Sequence
  Synthetic
ccgggagctgcatgtgtcagagg

SEQ ID NO: 69 - protein, 47 aa, Homo sapiens
FIIQCDLCLKWRVLPSSTNYQEKEFFDIWICANNPNRLENSCHQVEC

SEQ ID NO: 70 - protein, 47 aa, Homo sapiens
TTIQCDLCLKWRTLPFQLSSVEKDYPDTWVCSMNPDPEQDRCEASEQ

SEQ ID NO: 71 - protein, 51 aa, Arabidopsis thaliana
FTVQCASCFKWRLMPSMQKYEEIREQLLENPFFCDTAREWKPDISCDVPAD

SEQ ID NO: 72 - protein, 42 aa, Arabidopsis thaliana
YAAQCWKCLKVRSIESQEDYEEIRSKTLEKFFECKRCEEPGD

SEQ ID NO: 73 - protein, 47 aa, Arabidopsis thaliana
FAVQCEKCMKWRKIDTQDEYEDIRSRVQEDPFFCKTKEGVSCEDVGD

SEQ ID NO: 74 - protein, 47 aa, Arabidopsis thaliana
YAAQCDNCHKWRVIDSQEEYEDIRSKMLEDPFNCQKKQGMSCEEPAD

SEQ ID NO: 75 - protein, 46 aa, Arabidopsis thaliana
YWAQCESCEKWRLLPYDLNTEKLPDKWLCSMQTWLPGMNHCGVSEE

SEQ ID NO: 76 - protein, 46 aa, Arabidopsis thaliana
HWVACDKCGKWRLLPFGVFPEDLPEKWMCTMLNWLPGVNYCNVPED

SEQ ID NO: 77 - protein, 43 aa, Arabidopsis thaliana
RWATCDDCSKWRRLPVDALLSFKWTCIDNVWDVSRCSCSAPEE

SEQ ID NO: 78 - protein, 43 aa, Arabidopsis thaliana
QWVQCDACGKWRQLPVDILLPPKWSCSDNLLDPGRSSCSAPDE

SEQ ID NO: 79 - protein, 45 aa, Homo sapiens
TWVQCDECLKWRKLPGKIDPSMLPARWFCYYNSHPKYRRCSVPEE

SEQ ID NO: 80 - protein, 43 aa, Homo sapiens
TWVQCDACLKWRKLPDGMDQLPEKWYCSNNPDPQFRNCEVPEE

SEQ ID NO: 81 - protein, 47 aa, Homo sapiens
VWVQCSFPNCGKWRRLCGNIDPSVLPDNWSCDQNTDVQYNRCDIPEE

SEQ ID NO: 82 - protein, 43 aa, Arabidopsis thaliana
IWMQCDSCSKWRIIDEGVSVTGSAWFCSNNNDPAYQSCNDPEE

SEQ ID NO: 83 - protein, 46 aa, Arabidopsis thaliana
AWVRCDDCFKWRRIPASVVGSIDESSRWICMNNSDKRFADCSKSQE

SEQ ID NO: 84 - protein, 48 aa, Homo sapiens
VWVQCENENCLKWRLLSSEDSAKVDHDEPWYCFMNTDSRYNNCSISEE

SEQ ID NO: 85 - protein, 51 aa, Arabidopsis thaliana
NWLQCREEDTNGVICGKWRRAPRSEVQTKDWECFCCFSWDPSRADCAVPQK

SEQ ID NO: 86 - protein, 53 aa, Homo sapiens
YWVQCTKPECRKWRQLTKEIQLTPQIAKTYRCGMKPNTAIKPETSDHCSLPED

SEQ ID NO: 87 - protein, 84 aa, Arabidopsis
SYSTESAWVRCDDCFKWRRIPASVVGSIDESSRWICMNNSDKRFADCSKSQEMSNEEINEELGIGQDEADAYDCDAAKRGKEKE

SEQ ID NO: 88 - protein, 75 aa, Ricinis
HLPMDNAWVRCDECLKWRRIPVALVDSIGQTNCHWICKDNMDKAFADCSISQEKSNAEINAELGLSDADEDACDV

SEQ ID NO: 89 - protein, 76 aa, Oryza
SVPPAPAWVCCDDCEKWRCIPTELADKISKENLRWTCKENEDKTFANCSIPQEKTDDEINAELGLSDASADEANGD

SEQ ID NO: 90 - protein, 85 aa, Physcomitrella
SIDSKAGWVKCDDCKKWRSIPVELAEKIGDTNVYWSCKENPNKQFADCSIPQVKSNEEINRDLGISESSFCDEDEDGKEDETCDS

SEQ ID NO: 91 - protein, 85 aa, Physcomitrella
SINSKAGWVKCDDCNKWRSIPVDLAEKIGETNAYWSCKENPNKQFADCSIPQVKSNEEINRDLGISESSLCNEDEDEEHDETCDS

SEQ ID NO: 92 - protein, 73 aa, Chlamydomonas
PGETEQNWVLCDSCTKWRRVPAPYANSLDEDASWYCALNPNKAFASCSVPQELTDEQIDAGGDSGGESELDEE

SEQ ID NO: 93 - protein, 68 aa, Micromonas
TWVQCDSCEVWRRVPNYVSERLGDNDQWFCSLNNDPKFANCEAPQEIDNDEIDILLAAQEARQEAAEV

SEQ ID NO: 94 - protein, 74 aa, Ostreococcus
AWVQCDSCETWRRVPRVVADRLGENTAWFCHQNLDQKFSTCDDAQLSDDEIDRLMRAQAHAATSAANGEDACDG

(19) United States
(12) Patent Application Publication
(10) Pub. No.: US 2014/0148355 A1
(43) Pub. Date: May 29, 2014

(54) COMPOSITIONS AND METHODS FOR ANALYSIS OF H3K4 METHYLATED CHROMATIN

(71) Applicants: Reidunn B. Aalen, Oslo (NO); Tage Thorstensen, Oslo (NO); Rein Aasland, Bergen (NO); Verena Hoppmann, Albbruck (DE)

(72) Inventors: Reidunn B. Aalen, Oslo (NO); Tage Thorstensen, Oslo (NO); Rein Aasland, Bergen (NO); Verena Hoppmann, Albbruck (DE)

(73) Assignee: UNIVERSITY OF OSLO, Oslo (NO)

(21) Appl. No.: 14/007,677

(22) PCT Filed: Mar. 28, 2012
(86) PCT No.: PCT/IB2012/000807

§ 371 Date: Feb. 11, 2014

Related U.S. Application Data
(60) Provisional application No. 61/468,265, filed on Mar. 28, 2011.

(51) Int. Cl. G01N 33/53; C12Q 1/68; C07K 14/415

(52) U.S. Cl. CPC G01N 33/5308; C07K 14/415; C12Q 1/6809
USPC 506/9; 530/370; 435/252.3; 435/254.2; 435/325; 435/348; 435/252.33; 435/252.31; 435/252.34; 435/252.35; 435/254.21; 435/358; 435/365; 435/367; 435/369; 435/357; 435/352; 435/320.1; 536/23.4; 435/6.11; 435/455; 435/471

(57) ABSTRACT

The present invention relates to polypeptides that bind to H3K4 methylated chromatin, and in particular to the use of reagents comprising such polypeptides for epigenetic/epigenomic analysis.

### CROSS-REFERENCE TO RELATED APPLICATIONS

This application claims priority to provisional application 61/468,265, filed Mar. 28, 2011, which is herein incorporated by reference in its entirety.

### FIELD OF THE INVENTION

The present invention relates to polypeptides that bind to H3K4 methylated chromatin, and in particular to the use of reagents comprising such polypeptides for epigenetic/epigenomic analysis.

### BACKGROUND OF THE INVENTION

Transcription of eukaryotic genes is not only dependent on transcription factors facilitating the recruitment of the RNA polymerase pre-initiation complex, but also on the state of the template—the chromatin. The transcription machinery and most transcription factors can not access promoters and enhancers when the DNA is wrapped in nucleosomes and must be aided by factors changing the chromatin structure, e.g., ATP-dependent remodelling enzymes that remove or shift the position of nucleosomes along DNA, and histone-modifying enzymes.

A number of histone tail modifications have been identified including acetylation, phosphorylation, ubiquitination, and methylation (Spotswood & Turner, J Clin Invest 110: 577-582 2002), and some of these modifications serve as marks in chromatin that reflect the state of gene activity. Histone acetylation and methylation on lysine 4 of histone H3 (H3K4) are generally associated with active loci, while histones methylated on H3K9, H3K27 and H4K20 correlate with silenced chromatin (Kouzarides, Cell 128: 693-705 2007).

Unique combinations of histone modifications mark different genic and chromatin regions, implicating cross-talk between different modifications. This in correlation with different states of gene expression, is known as the histone code (Strahl & Allis, Nature 403: 41-45 2000). Histone modifying enzymes “write” the histone code, while the concept of this code in addition implies the existence of proteins that “read” these modifications and translate the embedded information into effects on chromatin structure and/or the transcription machinery (Turner, Nat Cell Biol 9: 2-6 2007). This prediction has indeed been borne out by the identification of a growing number of nuclear proteins that are fretted with one or more small histone recognition modules (Taverna et al, Nat Struct Mol Biol 14: 1025-1040 2007). Prominent examples are bromodomains, specific for acetylated lysines; chromodomains, that can bind H3K9me or in plants H3K27me3; and PHD fingers that recognize H3K4me3 or, in some cases acetylated or unmethylated histone tails (Chakravarty et al, Structure 17: 670-679 2009; Zeng et al, Nature 466: 258-262 2010). Several MBT domains have been shown to bind mono- and/or di-methylated lysines on both H3 and H4, but with less sequence selectivity than the chromodomains and PHD fingers (Bonasio et al, Semin Cell Dev Biol 21: 221-230 2010). A remarkable feature of histone recognition modules is that they often occur in a combinatorial fashion, either as multiple domains within one polypeptide or on different subunits of larger protein complexes, facilitating the simultaneous recognition of different histone modifications in chromatin (Ruthenburg et al, Nat Rev Mol Cell Biol 8: 983-994 2007).

The concerted action of “readers” and “writers” of a particular histone modification can explain how patterns of histone modifications can be propagated and inherited through many cell divisions, giving rise to the phenomenon of epigenetic inheritance. Similar mechanisms can explain how the transcriptional status of genes can be changed: A chromatin modifying enzyme could “read” one modification while “writing” another, i.e. by adding or alternatively, removing specific histone modifications. Furthermore, alterations of histone modification patterns may be brought about by sequence-specific transcription factors and/or non-coding RNAs that recruit different histone modifying enzymes as cofactors (coactivators, corepressors, or silencing complexes) (Goodman & Smolik, Genes Dev 14: 1553-1577 2000; Imhof, Brief Funct Genomic Proteomic 5: 222-227 2006; Muller & Kassis, Drosophila. Curr Opin Genet Dev 16: 476-484 2006; Ringrose & Paro, Development 134: 223-232 2007).

Histone lysine methylation is conferred by SET-domain proteins that can be divided into several evolutionarily conserved classes (Baumbusch et al, Nucleic Acids Res 29: 4319-4333 2001; Kouzarides, 2007 surpa; Wu et al, PloS one 5: e8570 2010) including: (1) the E(Z) class, involved in the maintenance of a transcriptionally repressive state of genes via H3K27 trimethylation; (2) SU(VAR)3-9 proteins, implicated in heterochromatinization via H3K9 methylation; (3) the TRXSET1 family that contribute to the active state via H3K4me3, and (4) ASH1 proteins associated with transcriptional elongation via H3K36me. In addition to the identity of the modified lysine, the number of methyl groups added is functionally significant (Fischer et al, J Plant Physiol 163: 358-368 2006). For example, using genome-wide chromatin profiling in mammalian cells, it was recently shown that, while H3K4me2 and me3 marks are prominent near transcription start sites (TSS), tissue-specific enhancers are enriched for monomethylated H3K4 (Heintzman et al, Nature 459: 108-112 2009; Heintzman et al, Nat Genet. 39: 311-3182007; Kim et al, Nature 465: 182-187 2010). In the model plant Arabidopsis thaliana, H3K4me3 is preferentially found in the 5′-end of highly expressed genes with low tissue-specificity, while H3K4me1 is highly correlated with CpG methylation in the transcribed region of genes (Zhang et al, Genome biology 10: R62 2009). Furthermore, H3K36 trimethylation of MADS box genes involved in flowering-time control and flower development, shows a positive correlation with transcription, but H3K36me1 does not (Grini et al, PloS one 4: e7817 2009; Xu et al, Mol Cell Biol 28: 1348-1360 2008).

Several SET domain histone methyltransferases (HMTases) have histone recognition modules as co-domains, either on the same polypeptide or on another subunit in a protein complex. Examples are: chromodomains in animal SU(VAR)3-9 proteins and PHD fingers in Trx/MLL proteins. Co-domains are thought to contribute to the recruitment of the histone modifiers to relevant sites in chromatin (Ruthenburg et al, 2007, supra). Alternatively, they may modulate the activity of the methyltransferase, as in the case for E(z)/EZH proteins where the H3K27me3-binding EED/Esc subunit contribute to H3K27me3 methylation on adjacent nucleosomes (Margueron et al, Nature 461: 762-767 2009).

While the functional role of several recognition modules has been worked out in some detail, how they contribute to maintaining or altering chromatin structure and thereby modulating gene expression is still unknown.

Thus, additional methods for analyzing methylated chromatin are needed.

### SUMMARY OF THE INVENTION

The present invention relates to polypeptides that bind to H3K4 methylated chromatin, and in particular to the use of reagents comprising such polypeptides for epigenetic/epigenomic analysis.

Accordingly, in some embodiments, the present invention provides isolated polypeptides comprising a CW domain operably linked to first member of a specific binding pair. In some embodiments, the CW domain is selected from the group consisting of Arabidopsis ASHH2, AtMDB1, AtMBD2, AtMBD3, AtMBD4, VAL1, VAL2, NP_{—}179516, FB304-ARATH, NP_{—}191849, O23424_ARATH CW domains and human and mouse ZCWPW1, ZCWPW2, MORC1, MORC2, MORC3 CW domains. In some embodiments, the CW domain is an Arabidopsis ASHH2 CW domain. In some embodiments, the first member of a specific binding pair is a protein tag. In some embodiments, the protein tag is selected from the group consisting of glutathione-S-transferase (GST), a His-tag, a maltose binding protein-tag, a SBP-tag, a Flag-tag, a HA-tag, and a Myc-tag.

In some embodiments, the present invention provides a nucleic acid encoding an isolated polypeptide as described above. In some embodiments, the present invention provides an expression vector comprising the nucleic acid as just described. In some embodiments, the present invention provides a host cell comprises and expresses the nucleic acids or vectors of the present invention.

In some embodiments, the present invention provides systems or kits for analysis of methylation of chromatin comprising: a polypeptide comprising a CW domain operably linked to a first member of a specific binding pair; and at least one reagent comprising a second member of said specific binding pair. In some embodiments, the CW domain is a selected from the group consisting of Arabidopsis ASHH2, AtMDB1, AtMBD2, AtMBD3, AtMBD4, VAL1, VAL2, NP_{—}179516, FB304-ARATH, NP_{—}191849, O23424_ARATH CW domains and human and mouse ZCWPW1, ZCWPW2, MORC1, MORC2, MORC3 CW domains. In some embodiments, the CW domain is an Arabidopsis ASHH2 CW domain. In some embodiments, the first member of a specific binding pair is a protein tag. In some embodiments, the protein tag is selected from the group consisting of glutathione-S-transferase (GST), a His-tag, a maltose binding protein-tag, a SBP-tag, a Flag-tag, a HA-tag, and a Myc-tag. In some embodiments, the reagent comprising a second member of said specific binding pair comprises a media support. In some embodiments, the media support is selected from the group consisting of magnetic beads, a polymeric beads, planar supports, and chromatography supports. In some embodiments, the reagent comprises a second member of said specific binding pair comprises a member selected from the group consisting of glutathione, amylase, Ni, avidin, and an antibody specific for FLAG, HA or myc.

In some embodiments, the present invention provides methods for analyzing methylation of chromatin comprising: contacting a chromatin sample with a reagent comprising a CW domain polypeptide to form a reagent-chromatin complex; and analyzing said reagent-chromatin complex. In some embodiments, the analyzing comprises isolating said reagent-chromatin complex. In some embodiments, the reagent further comprises a first member of a specific binding pair and said isolating further comprises contacting said complex with a reagent comprising a second member of said specific binding pair. In some embodiments, the analyzing further comprises analysis of nucleic acid sequences associated with said chromatin.

In some embodiments, the present invention provides isolated polypeptides comprising a CW domain operably linked to an effector domain polypeptide. In some embodiments, the CW domain is selected from the group consisting of Arabidopsis ASHH2, AtMDB1, AtMBD2, AtMBD3, AtMBD4, VAL1, VAL2, NP_{—}179516, FB304_ARATH, NP_{—}191849, O23424_ARATH CW domains and human and mouse ZCWPW1, ZCWPW2, MORC1, MORC2, MORC3 CW domains. In some embodiments, the CW domain is an Arabidopsis ASHH2 CW domain. In some embodiments, the effector domain polypeptide reacts with DNA or nucleosomal histones. In some embodiments, the present invention provides a nucleic acid encoding an isolated polypeptide as described above. In some embodiments, the present invention provides an expression vector comprising the nucleic acid as just described. In some embodiments, the present invention provides a host cell comprises and expresses the nucleic acids or vectors of the present invention. In some embodiments, the present invention provides a transgenic organism comprising the vectors. In some embodiments, the present invention provides methods for altering the chromatin of a cell or organism comprising: introducing the vectors into a target cell or organism.

Additional embodiments are described herein.

### DESCRIPTION OF THE FIGURES

FIG. 1 Histone tail methylation in chromatin of selected genes from wild type and ashh2-1 mutant seedlings. ChIP using antibodies for (A) H3K4me3, (B) H3K36me3, (C) H3K4me1 and (D) results without antibody (-ab). Data are shown as percent of input. The Ta3 retrotransposon was used as reference. Standard deviations are shown. The FLC C primers amplify a region in the first exon of the gene, and FLC E in the first intron. TSS indicate primers near the transcriptional start site, while other primers used are in the body of the genes.

FIG. 2 Properties of genes with changed expression levels in ashh2 seedlings. (A) Functional annotation using gene onthology (GO) of genes differentially expressed in ashh2-1 inflorescences relative to wt. (B, C) Distribution of H3K4me marks for genes down-regulated in ashh2 mutant seedlings (cf. Table III) and genes from wt seedlings according to (Zhang et al., 2009). In (B) genes without any H3K4 methylation are included, in (C) genes without H3K4 methylation were not included. ‘N’: number of genes.

FIG. 3 The ASHH2 CW domain binds histone H3 tails methylation on lysine 4. (A) Cartoon of the ASHH2 protein with CW, AWS and KMT (SET domain) indicated. The two recombinant proteins used in this study are also illustrated. (B) SDS-PAGE of purified GST and GST-ASHH2-CW. (C) Pull-down assay using biotinylated histone peptides and recombinant GST, GST-ASHH2-CW and GST-ING2-PHD as indicated. The ratio of proteins and peptides were 0.5 μg:0.5 μg. (D) Pull-down assay using biotinylated histone peptides and recombinant GST and GST-ASHH2-CW as indicated. The ratio of proteins and peptides were 1.0 μg:0.5 μg. (E) Surface plasmon resonance traces from an experiment with immobilised, biotinylated H3K4me1 peptide and GST-ASHH2-CW. (F) Dissociation constants for GST-ASHH2-CW and the three H3K4me peptides indicated calculated from surface plasmon resonance data. The number of replicates are indicated.

FIG. 4 CW domains from four different proteins bind methylated H3K4 peptides. (A) Cartoons of the proteins studied: human MORC4, Arabidopsis VAL1, and human ZCWPW1. (B, C) Pull-down assay using biotinylated histone peptides and (B) recombinant GST, GST-Val1-CW and GST-MORC4-CW or (C) GST, GST-ASHH2-CW, and GST-ZCWPW1-CW, as indicated. The ratio of proteins and peptides were equimolar.

FIG. 5 Solution structure of the CW domain and prediction of its binding site for the histone tail. (A) Backbone traces of 20 conformers of the solution structure of the ASHH2 CW domain showing residues 858-928. Secondary structure elements are indicated in blue (b-strands), red (a-helix), and pink (helix-like segments). The zinc ion is shown in green, and the two tryptophans of the aromatic cage are shown in purple sticks. (B) Spheres view of the ASHH2 CW domain highlighting hydrophobic residues (yellow), side chain carboxyl groups of glutamates and aspartates (red), nitrogens of the side chains of lysine and arginine (blue), and the zinc ion (green). The two conserved tryptophans forming the aromatic cage are shown in orange. A cartoon trace in dark blue is shown in the background. (C) Surface representations and surface potential of the ASHH2 CW domain. Negative and positive potentials are shown in shades of red and blue, respectively. The predicted placement of the histone tail backbone and the aromatic cage are indicated by a dotted line and a circle, respectively. (D) Structure of the ZCWPW1 CW domain (pdb:2e61) colour coded as in (B). (E) ¹H- and ¹⁵N-amide chemical shift changes for ASHH2 CW bound to the H3K4me1 peptide. The secondary structure elements are indicated in the same colour code as in (A).

FIG. 6 NMR spectroscopy of the ASHH2 CW domain and its complex with a histone H3K4me1 peptide. ₁₅N-₁H HSQC spectrum of the CW domain in complex with the histone H3K4me1 peptide (1-15). Chemcial shift assignments of the backbone residues are shown.

FIG. 7 Probing the predicted histone tail binding site with mutant CW domains. (A) A space-filling model of the ASHH2 CW domain is shown with residues mutated highlighted: grey: loss of binding; light grey: reduced/altered binding. The predicted binding site for the histone tail is indicated as in FIG. 6D. (B and C) Pull-down assays using biotinylated histone peptides and recombinant GST and GST-ASHH2-CW mutant proteins as indicated. The ratio of proteins and peptides were 1.0 μg:0.5 μg.

FIG. 8 Chromatin pulldown (ChPD) using the ASHH2 CW. (A) ChPD with nucleosomes (from chromatin as prepared for ChIP from Arabidopsis seedlings) and immobilised GST, GST-CW_{ASHH2}, GST-CW_{ASHH2}-W874A (non-binding mutant), and GST-WIYLD_{SUVR4} (negative control) as indicated. Modified histones were visualised by western blot using antibodies as indicated. Molecular size markers are shown to the right. (B) qPCR with primers for assumed ASHH2 target genes on DNA isolated from seedling chromatin pulled down with GST-CW_{ASHH2}. ChPD with GST alone was used as a negative control. Data are shown as percent of input. The Ta3 retrotransposon was used as reference. Standard deviations are shown. (C) ChIP using antibodies against H3K4me1, H3K4me3 and H3K36me3 and primers for the same genes as in (B). Data are shown relative to Ta3. (D) ChPD from wt and ashh2 mutant seedlings as in (A) using antibodies as indicated. ChPD with GST alone was used as a negative control and histone H3 as reference.

FIG. 9 Multiple sequence alignment of the human and Arabidopsis CW domains and plant ASHH2 orthologs. Multiple sequence alignments of (A) human and plant CW domains and (B) selected plantorthologs of ASHH2 CW domains. Note that in (B), the family-specific C-terminal extension for plant ASHH2 CW domains is included. The alignments were generated with Muscle and manually edited and colour coded with the ClustalX colour scheeme in Jalview. The conserved cysteines involved in zinc ion binding, β-strands, the α1-helix, and the three helix-like elements are indicated below the alignment. The sequence numbering for the ASHH2 CW domain is shown at the top. An unrooted tree made by the neighbour-joining method (in Jalview) is shown to the left.

FIG. 10 A model for ASHH2 function and the role of its CW domain. The CW domain of ASHH2 binds H3K4me2 found close to transcription start site (TSS) in both highly expressed house-keeping genes and weakly expressed tissue-specific genes, but not in silent genes. In addition CW binds H3K4me1 found along the transcribed gene regions. This facilitates H3K36me3 methylation both near the TSS, and along the gene body. Loss of H3K36me3 has little consequences for the transcription level of highly expressed house-keeping genes. However, loss of H3K36me3 due to mutation in ASHH2 results in non-sustainable transcription of genes that may carry both active and repressive chromatin marks, e.g. K4me and H3K27me3, and/or low and tissue-specific expression. Due to the lack of H3K4me marks ASHH2 is not active on silent genes and thus there is no effect on transcription when ASHH2 is mutated. Icons for each of the histone modifications are shown to the right. The ASHH2 protein and its SET domain are shown in green, while the CW domain engaged in H3K4me-binding is shown in yellow. RNA polymerase II occupancy is illustrated with blue pentagons, while the extent of transcription is indicated with arrows.

### DEFINITIONS

To facilitate an understanding of the present invention, a number of terms and phrases as used herein are defined below:

The term “CW domain polypeptide” refers to a polypeptide comprising a CW domain consensus sequence. Exemplary CW domain polypeptide sequences are provided as SEQ ID NOs:1-28.

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid.

The term “fusion” when used in reference to a polypeptide refers to a chimeric protein containing a protein of interest joined to an exogenous protein fragment (the fusion partner). The fusion partner may serve various functions, including enhancement of solubility of the polypeptide of interest, as well as providing an “affinity tag” to allow purification of the recombinant fusion polypeptide from a host cell or from a supernatant or from both. If desired, the fusion partner may be removed from the protein of interest after or during purification.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. An example of a protein domain is the CW domain.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid, and is referred to using the functional term “substantially homologous”. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith & Waterman [1981] Adv. Appl. Math., 2:482) by the homology alignment algorithm of Needleman and Wunsch (Needleman & Wunsch [1970] J. Mol. Biol., 48:443), by the search for similarity method of Pearson and Lipman (Pearson & Lipman [1988] Proc. Natl. Acad. Sci. U.S.A., 85:2444), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al. [1987] Science 236:1237). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” refer to a DNA sequence that is located at the 5′ end (i.e. precedes) of the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

The term “regulatory region” refers to a gene's 5′ transcribed but untranslated regions, located immediately downstream from the promoter and ending just prior to the translational start of the gene.

The term “promoter region” refers to the region immediately upstream of the coding region of a DNA polymer, and is typically between about 500 bb and 4 kb in length, and is preferably about 1 to 1.5 kb in length.

The term “vector” refers to nucleic acid molecules that transfer DNA segment(s) from one cell to another. The term “vehicle” is sometimes used interchangeably with “vector.”

The terms “expression vector” or “expression cassette” refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The term “purified” refers to molecules, either nucleic or amino acid sequences, that are removed from their natural environment, isolated or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the term “purified” or “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

### DETAILED DESCRIPTION OF THE INVENTION

The present invention relates to polypeptides that bind to H3K4 methylated chromatin, and in particular to the use of reagents comprising such polypeptides for epigenetic/epigenomic analysis.

Experiments conducted during the course of development of embodiments of the present invention identified the CW domain as a new type of histone recognition module and explored its properties. This domain, named after its conserved cysteine and tryptophan residues, was first identified as an MBD-associated domain (MAD) in a subgroup of methyl-CpG-binding proteins of Arabidopsis (Berg et al, 2003). The CW domain is found in a small number of chromatin-related proteins in animals and plants (Perry & Zhao, 2003; see Table I). Some of the genes that encode CW proteins have mutant alleles with phenotypes that underscore their functional importance: Mutation in the mouse Morc1 causes arrested spermatogenesis (Inoue et al, 1999), Morc2b was recently shown to be involved in hybrid sterility (Mihola et al, 2009), and MORC4 has been found highly expressed in large B-cell lymphomas (Liggins et al, 2007). The Arabidopsis val1val2 double mutant fail to repress embryonic development during vegetative growth (Suzuki et al, 2007). The mammalian CW protein AOF1/LSD2 (alias KDM1B) is a H3K4me1 and me2-specific histone demethylase (Karytinos et al, 2009). AOFULSD2 has a demethylase-independent repressor function, which, on the other hand, requires the CW domain (Yang et al, 2010).

The best studied CW protein is, however, the Arabidopsis ASH1 HOMOLOG2 (ASHH2), also known as SDG8/EFS/CCR1. ASHH2 is a ˜200K SET-domain protein considered to be a major H3K36me2/me3 HMTase in Arabidopsis, as chromatin of ashh2 mutants shows a global reduction in H3K36me2/me3 levels (Xu et al, 2008; Zhao et al, 2005). In ASHH2, a CW domain precedes the AWS and SET domains. Mutations in ASHH2 confer pleiotropic effects like small, bushy plants with early flowering, homeotic changes of floral organs, and severely reduced fertility. The expression of the major regulator of flowering time in Arabidopsis, FLOWERING LOCUS C (FLC), a direct target of ASHH2 (Ko et al., 2010), as well as other transcription factor genes involved in these developmental processes, is repressed in the mutant correlating with a reduction in H3K36me2/me3 levels in mutant plants (Dong et al, 2008; Grini et al, 2009; Kim et al, 2005; Xu et al, 2008; Zhao et al, 2005). It is not clear, however, whether this mark is a prerequisite for gene expression.

In vitro, ASHH2 is active on histone H3 isolated from eukaryote nuclei, but not on recombinant histones (Dong et al, 2008; Grini et al, 2009), indicating the requirement of a pre-modified histone tail.

### 1. CW Domain Compositions

The present invention takes advantage of a newly discovered binding activity of the CW domain. Specifically, it has been found that the CW domain (present in a number of proteins) binds to methylated chromatin with high affinity. Examples of CW domain polypeptides suitable for use in the present invention, included, but are not limited to, polypeptides comprising the CW domains listed in Table I and SEQ ID NOs:1-28 and polypeptides that are homologous thereto.

TABLE I
Exemplary protein with CW domains

| | | Chromosomal | |
|---|---|---|---|
| | | Location (Hs) | |
| Protein family | Protein | GeneID (At) | Function/phenotype |
| Human | | | |
| CW + PWWP | ZCWPW1 | 7q22.1 | |
| | ZCWPW2 | 3p24.1 | |
| MORC family | MORC1 | 3q13 | Mouse: mitochondria |
| HSP90-like | | | (spermatogenesis) |
| ATPases | MORC2 | 22q12.2 | Mouse: induced by Prdm9, |
| | | | A hybrid sterility gene |
| | MORC3 | | Mouse; CW domain |
| | | | Required for proper |
| | | | Localization in the |
| | | | nucleus |
| LSD1 | AOF1(LSD2) | 6p22.3 | H3K4me 1,2-specific |
| Histone | | | Histone demethylase |
| Demethylases | | | |
| Arabidopsis | | | |
| MBD family | AtMDB1 | At4g22745 | Euchromatic localization |
| (CW-MBD) | AtMBD2 | At5g35330 | |
| | AtMBD3 | At4g00416 | |
| | AtMBD4 | At3g63030 | |
| ASHH family | ASHH2 | At1g77300 | Histone methyltransferase |
| Histone | | | (H3K36me2,3); severe |
| Methyltransferase | | | pleiotropic phenotype; |
| (CW + SET) | | | small organs, early |
| | | | Flowering, distorted |
| | | | Development of |
| | | | Reproductive organs |
| VP1/ABI3-Like | VAL1 | At2g30470 | Repressers of embryonic |
| (B3 + CW) | | | genes at germination |
| | VAL2 | At4g32010 | Double mutant with |
| | | | Embryonic seedling |
| | | | Phenotypes |
| PHD + CW | NP_179516 | At2g19260 | |
| F-box proteins | FB304_ARATH | At3g54460 | |
| SWI/SNF-ATPase | | | |
| Putative chromatin | | | |
| Remodeling | | | |
| Solo CW | NP_191849 | At3g62900 | |
| | O23424_ARATH | At4g15730 | |

Arabidopsis thaliana NP_177854 (AT1G77300.1p)
(SEQ ID NO: 1)
WVRCDDCFKWRRIPASVVGSIDESSRWICMNNSDKRFADCSKSQEMS
Arabidopsis thaliana AAW70394.1 (At4g22745)
(SEQ ID NO: 2)
VQCEKCMKWRKIDTQDEYEDIRSRVQEDPFFCKTKEGVSCEDVGDLN
Arabidopsis thaliana ABF83690.1 (At5g35330)
(SEQ ID NO: 3)
TVQCASCFKWRLMPSMQKYEEIREQLLENPFFCDTAREWKPDISCDVPAD
IY
Arabidopsis thaliana NP_567177.1 (AT4G00416)
(SEQ ID NO: 4)
AAQCWKCLKVRSIESQEDYEEIRSKTLEKFFECKRCEEPGDMV
Arabidopsis thaliana AAQ65139 (At3g63030)
(SEQ ID NO: 5)
AAQCDNCHKWRVIDSQEEYEDIRSKMLEDPFNCQKKQGMSCEEPADI
Arabidopsis thaliana AAB63089.1 At2g30470
(SEQ ID NO: 6)
WATCDDCSKWRRLPVDALLSFKWTCIDNVWDVSRCSCSAPEE
Arabidopsis thaliana NP_194929.2 At4g32010
(SEQ ID NO: 7)
WVQCDACGKWRQLPVDILLPPKWSCSDNLLDPGRSSCSAPDELS
Arabidopsis thaliana AAO64869.1 At2g19260
(SEQ ID NO: 8)
KHCDKPGTVEKMLICDECEEAYHTRCCGVQMKDVAEIDEWLCPSC
Arabidopsis thaliana At3g54460
(SEQ ID NO: 9)
WMQCDSCSKWRRIIDEGVSVTGSAWFCSNNNDPAYQSCNDPEELW
Arabidopsis thaliana NP_191849 At3g62900
(SEQ ID NO: 10)
WVACDKCGKWRLLPFGVFPEDLPEKWMCTMLNWLPGVNYCNVPEDET
Arabidopsis thaliana At4g15730 At4g15730
(SEQ ID NO: 11)
WAQCESCEKWRLLPYDLNTEKLPDKWLCSMQTWLPGMNHCGVSEEET
Human AAH02725.1 ZCWPW1
(SEQ ID NO: 12)
WVQCSFPNCGKWRRLCGNIDPSVLPDNWSCDQNTDVQYNRCDIPEETW
Mouse AAX39493.1 ZCWPW1
(SEQ ID NO: 13)
WVQCSSPKCEKWRQLRGNIDPSVLPDDWSCDQNPDPNYNRCDIPEESW
Human NP_001035522.1 ZCWPW2
(SEQ ID NO: 14)
WVQCENENCLKWRLLSSEDSAKVDHDEPWYCFMNTDSRYNNCSISEEDF
Mouse AAI25263.1 ZCWPW2
(SEQ ID NO: 15)
WVQCENESCLKWRLLSPAAAAAVNPSEPWYCFMNTDPSYSSCSVSEEDF
Human EAW79714.1 MORC1
(SEQ ID NO: 16)
QCDLCLKWRVLPSSTNYQEKEFFDIWICANNPNRLENSCHQVECLP
Mouse NP 034946.1 MORC1
(SEQ ID NO: 17)
QCDLCLKWRVLPSSSNYQEKGLPDLWICASNPNNLENSCNQIERLP
Human NP_055756.1 MORC2
(SEQ ID NO: 18)
TIQCDLCLKWRTLPFQLSSVEKDYPDTWVCSMNPDPEQDRCEASEQKQ
Mouse CAX16071.1 MORC2
(SEQ ID NO: 19)
TIQCDLCLKWRTLPFQLSAVEEGYPINWVCSMNPDPEQDQCEA
Human AAI32732.1 MORC3
(SEQ ID NO: 20)
WVQCDACLKWRKLPDGMDQLPEKWYCSNNPDPQFRNC
Mouse AAH26506.1 MORC3
(SEQ ID NO: 21)
VQCDACLKWRKLPDGIDQLPEKWYCSNNPDPQFRNC
Arabidopsis thaliana BAC43037
(SEQ ID NO: 22)
QCSAVNWLQCREEDTNGVICGKWRRAPRSEVQTKDWECFCCFSWDPSRAD
CAVPQELET
Arabidopsis thaliana NP_179516.2
(SEQ ID NO: 23)
QCSAVNWLQCREEDTNGVICGKWRRAPRSEVQTKDWECFCCFSWDPSRAD
CAVPQELET
Arabidopsis thaliana AAC16456.1
(SEQ ID NO: 24)
QCSAVNWLQCREEDTNGVICGKWRRAPRSEVQTKDWECFCCFSWDPSRAD
CAVPQK
Arabidopsis lyrata 9320066 XP_002886050.1
(SEQ ID NO: 25)
QCSAVNWLQCREEDSNGDICGKWRRAPRSEVQTKDWECFCCVFWDPSRAD
CAVPQELET
Zea mays NP_001152214.1
(SEQ ID NO: 26)
SIGNWIQCRETLNPGDSDKQVVCGKWRRAPLYVVQSDNWDCFCCLLWDPV
HADCAVPQEL
XP_002527977.1 Ricinis communis
(SEQ ID NO: 27)
SLSNWLQCQEVLYDETGEPIEGTKCRKWRRAPLSEVQTDEWDCSCSVTWD
PFHSDCAVPQ
EEC77272.1 Oryza sativa
(SEQ ID NO: 28)
SIGNWIQCREILSEGDSDKPVVCGKWRRAPLFVVQSDDWDCSCCLPWDPA
HADCAVPQEL

Embodiments of the present invention provides for the use of compositions (e.g., reagents) comprising CW domain polypeptides (and variants thereof) for a variety of uses, including but not limited to, isolation and/or identification of methylated chromatin and chromatin engineering. The compositions comprising CW domain polypeptides preferably comprise a CW domain that is least 50%, 60%, 70%, 80%, 90%, 95%, 98%, 99% or 100% identical to SEQ ID NOs: 1-28 and which bind to methylated chromatin. In some embodiments, the CW polypeptide consists solely of the CW domain, while in other embodiments, the CW polypeptide comprises amino acid sequence flanking the CW domain consensus sequence.

In some embodiments, the compositions comprising CW domain polypeptides further comprise a first member of a specific binding pair. The first member of a specific binding pair can preferably be a protein tag. Accordingly, the present invention provides fusion proteins comprising a CW domain polypeptide as described above in operably association with a protein tag. The protein tag can be located at either the N- or C-terminal of the CW domain polypeptide. Preferably, protein tags are polypeptide sequences that bind to a compound or another protein so that isolation of the tagged fusion protein is facilitated. Suitable protein tags include, but are not limited to, glutathione-S-transferase (GST), the His-tag (e.g., a polyhistidine tag of 5, 6, or 7 histidine residues), the maltose binding protein-tag, SBP-tag, and epitope tags such as the Flag-tag (e.g., N-DYKDDDDK-C) (SEQ ID NO: 29), the HA-tag, the Myc-tag, and the like. In these embodiments, the protein tag is the first member of a specific binding pair. The compound or protein that binds to the peptide tag is the second member of a specific binding pair, for example, glutathione for GST, Ni for a His-tag, antibodies for a FLAG-tag, antibodies for the HA-tag, antibodies for the myc-tag, amylase for the MPB-tag, streptavidin for the SBP-tag, etc.

In some embodiments, the first member of a specific binding pair is covalently attached to the CW domain polypeptide. The CW domain polypeptide can be directly modified or amino acids that are covalently modified can be incorporated into the CW domain polypeptide. Suitable first members of specific binding pair that can be covalently attached to a CW domain polypeptide include, but are not limited to, biotin and haptens such as dinitrophenyl (DNP)), biotin, fluorescein, digoxigenin and the like. In these embodiments, the second member of the specific binding pair is avidin in the case of biotin and specific antibodies in the case of the haptens.

In some embodiments, the compositions comprising CW domain polypeptides further comprise a chromatin effector domain. In some preferred embodiments, the chromatin effector domain is operably associated with the CW domain in a fusion protein. Suitable effector domains include catalytic domains that can modify DNA (e.g. DNA-methylation, -cleavage or -recombination) or nucleosomal histones (e.g. histone acetylation, methylation, or phosphorylation).

Accordingly, the present invention provides compositions comprising CW domain polypeptides and nucleic acid sequences that encode composition comprising CW domain polypeptides. Other embodiments of the present invention provide fusion proteins or functional equivalents of these CW domain polypeptides, as well as nucleic acids encoding such CW domain polypeptides and fusions (e.g., fusions with a protein tag or effector domain). In still other embodiments, the present invention provides CW domain polypeptide variants, homologs, and mutants. In some embodiments of the present invention, the polypeptide is a naturally purified product, in other embodiments it is a product of chemical synthetic procedures, and in still other embodiments it is produced by recombinant techniques using a prokaryotic or eukaryotic host (e.g., by bacterial, yeast, higher plant, insect and mammalian cells in culture).

The CW domain polynucleotides of the present invention may be employed for producing CW domain polypeptides (and fusion proteins) by recombinant techniques. Thus, for example, the CW domain polynucleotide may be included in any one of a variety of expression vectors for expressing a polypeptide. In some embodiments of the present invention, vectors include, but are not limited to, chromosomal, nonchromosomal and synthetic DNA sequences (e.g., derivatives of SV40, bacterial plasmids including derivatives of Agrobacterium tumefaciens Ti plasmids (T-DNA vectors), phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments of the present invention provide recombinant constructs comprising one or more of the sequences as broadly described above (e.g., SEQ ID NOS: 1-28), preferably in association with protein tag or effector domain as described above. In some embodiments of the present invention, the constructs comprise a vector, such as a plasmid or viral vector, into which a sequence of the invention has been inserted, in a forward or reverse orientation. In still other embodiments, the heterologous structural sequence (e.g., SEQ ID NOs:1-28) is assembled in appropriate phase with translation initiation and termination sequences. In preferred embodiments of the present invention, the appropriate DNA sequence is inserted into the vector using any of a variety of procedures. In general, the DNA sequence is inserted into an appropriate restriction endonuclease site(s) by procedures known in the art, or by employing site specific recombination using the Gateway cloning system.

Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); 2) Eukaryotic—pWLNEO, pSV2CAT, pOG44, PXT1, pSG (Stratagene) pSVK3, pBPV, pMSG, pSVL (Pharmacia); and 3) Baculovirus—pPbac and pMbac (Stratagene) and plant vectors including, but not limited to, Agrobacterium Ti plasmid-based vectors, especially Ti vectors and binary vectors, and plant virus vectors systems. Any other plasmid or vector may be used as long as they are replicable and viable in the host. In some preferred embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, the DNA sequence in the expression vector is operatively linked to an appropriate expression control sequence(s) (promoter) to direct mRNA synthesis. Promoters useful in the present invention include, but are not limited to, the LTR or SV40 promoter, the E. coli lac or trp, Cauliflower mosaic virus 35S promoter, the phage lambda P.sub.L and P.sub.R, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In some embodiments of the present invention, transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin by 100 to 270, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

In a further embodiment, the present invention provides host cells containing the above-described constructs. In some embodiments of the present invention, the host cell is a higher eukaryotic cell (e.g., a mammalian or insect cell). In other embodiments of the present invention, the host cell is a lower eukaryotic cell (e.g., a yeast cell). In still other embodiments of the present invention, the host cell can be a prokaryotic cell (e.g., a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, (Gluzman, Cell 23:175 [1981]), C127, 3T3, 293, 293T, HeLa and BHK cell lines.

The constructs in host cells can be used in a conventional manner to produce the gene product encoded by the recombinant sequence. In some embodiments, introduction of the construct into the host cell can be accomplished by calcium phosphate transfection, DEAE-Dextran mediated transfection, or electroporation (See e.g., Davis et al. [1986] Basic Methods in Molecular Biology). Alternatively, in some embodiments of the present invention, the polypeptides of the invention can be synthetically produced by conventional peptide synthesizers.

Proteins can be expressed in mammalian cells, yeast, bacteria, or plant cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al. (1989) Molecular. Cloning: A Laboratory Manual, Second Edition, Cold Spring Harbor, N.Y.

In some embodiments of the present invention, following transformation of a suitable host strain and growth of the host strain to an appropriate cell density, the selected promoter is induced by appropriate means (e.g., temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

The present invention also provides methods for recovering and purifying CW domain polypeptides from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In other embodiments of the present invention, protein-refolding steps can be used as necessary, in completing configuration of the mature protein. In still other embodiments of the present invention, high performance liquid chromatography (HPLC) can be employed for final purification steps.

As described above, the present invention also provides fusion proteins incorporating all or part of a CW domain polypeptide. Accordingly, in some embodiments of the present invention, the coding sequences for the polypeptide can be incorporated as a part of a fusion gene including a nucleotide sequence encoding a different polypeptide. Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation, or alternatively site specific recombination using the Gateway system. In another embodiment of the present invention, the fusion gene can be synthesized by conventional techniques including automated DNA synthesizers. Alternatively, in other embodiments of the present invention, PCR amplification of gene fragments can be carried out using anchor primers which give rise to complementary overhangs between two consecutive gene fragments which can subsequently be annealed to generate a chimeric gene sequence (See e.g., Current Protocols in Molecular Biology, supra).

In an alternate embodiment of the invention, CW domain polypeptides are synthesized, whole or in part, using chemical methods well known in the art. For example, peptides can be synthesized by solid phase techniques, cleaved from the resin, and purified by preparative high performance liquid chromatography (See e.g., Creighton (1983) Proteins Structures And Molecular Principles, W H Freeman and Co, New York N.Y.). In other embodiments of the present invention, the composition of the synthetic peptides is confirmed by amino acid analysis or sequencing (See e.g., Creighton, supra). Direct peptide synthesis can be performed using various solid-phase techniques (Roberge et al. [1995] Science 269:202) and automated synthesis may be achieved. Additionally, the amino acid sequence of a CW domain polypeptide, or any part thereof, may be altered during direct synthesis and/or combined using chemical methods with other sequences to produce a variant polypeptide.

### 2. Uses of CW Domain Compositions

The CW domain compositions of embodiments of the present invention have a variety of uses. For example, CW domain polypeptides associated with a first member of a specific binding pair can be can be used to isolate methylated chromatin (for example, H3K4 methylated chromatin) and can therefore substitute for antibodies with the same specificity and be used as an alternative to chromatin immunoprecipitation. The purified chromatin can thereafter be used to identify methylated (e.g., H3K4me marked) genes and enhancers and also additional histone or DNA-methylation marks and macromolecules associated with methylated (e.g., H3K4me-marked) chromatin. The CW domain compositions of the present invention have submicromolar affinity for methylated chromatin and thus represent a substantial improvement over current reagents that utilize antibodies for analysis of methylated chromatin. In some preferred embodiments, the CW domain polypeptide compositions comprising the ASHH2 CW domain (SEQ ID NO:1) are used to isolate monomethylated H3K4.

Accordingly, in some embodiments, the present invention provides systems, kits and methods for analysis of the methylation status or chromatin associated with a particular chromosomal locus or gene. In these embodiments, a sample containing chromatin is contacted with CW domain polypeptide composition comprising a first member of a specific binding pair. In some preferred embodiments, the chromatin is crosslinked, for example by incubation with formaldehyde or DTBP. In some embodiments, following crosslinking, the cells are lysed and the DNA is broken into pieces (e.g., 0.2-1 kb in length) by sonication. The CW domain polypeptide composition comprising a first member of a specific binding pair is preferably incubated with the chromatin under conditions suitable for binding of the CW domain polypeptide to the methylated chromatin so that a CW domain-chromatin complex is formed. The complex can then isolated by contacting the solution containing the complex with a media comprising a second member of the specific binding pair under conditions suitable for binding of the first and second members of the specific binding pair. Suitable media include, but are not limited to, magnetic beads, polymeric beads, planar supports such as plastic or glass slides, and chromatography supports that display the second member of the specific binding pair. The bound complex can then be analyzed on the media or eluted from the media and subjected to further analysis.

In some embodiments, the nucleic acid associated with the isolated chromatin is analyzed. Methods for analysis include, but are not limited to sequencing the nucleic acid, hybridization with nucleic acid probes, and array hybridization assays.

In other embodiments, vectors encoding CW domain compositions comprising chromatin effector domains are used to express the CW domain composition in a biological target, for example a cell, bacteria, fungi, plant or animal. The effect of modification of chromatin by the effector domain is then assessed.

### Experimental

The following examples are provided in order to demonstrate and further illustrate certain preferred embodiments and aspects of the present invention and are not to be construed as limiting the scope thereof.

### Materials and Methods

### Microarray Experiment and Data Analysis

Experiments were performed basically as in Grini et al (2009) using five biological replicas with 12 plants each, of both Arabidopsis thaliana plants Col ecotype and the ashh2-1 mutant (identical to sdg8-1) (Grini et al, 2009, supra). 8-10 days old seedlings of each biological repeat were harvested in bulk at same developmental stage and total RNA was extracted using RNeasy midi kit (Qiagen).

Gene expression profiles for ashh2 inflorescences were compared to their wt counterparts using two-color microarrays and statistical analysis according to Kusnierczyk et al (2007). Differentially expressed genes were identified using the limma software package (Smyth, 2004). All parts of the data analysis were performed using R (R Development Core Team, 2007). All data are MIAME compliant and that the raw data has been deposited in the MIAME compliant database GEO.

The microarray data was compared with those of Cazzonelli et al Plant Cell 21: 39-53 (2009) and Xu et al (2008), supra. The mutant alleles and conditions for these two sets were green rosette leaves from 10 days old ccr1-1 seedlings using ATH1 Arrays (Affymetrix) (Cazzonelli et al, 2009, supra), and 6 days old sdg2-1 and sdg2-2 seedlings analyzed on the CATMA 24k chip (Xu et al, 2008). The gene lists were generated with log 2 absolute value 0.7 for ashh2-1 (P<0.01), 0.8 for sdg8-2 (P<0.05), and 0.3 for ccr-1 (P<0.05). The TAIR GO annotation tool was used to assign functional classes to the genes.

Real-Time Quantitative PCR (qPCR)

qPCR was basically performed as in Grini et al (2009), supra. Expression levels of target genes in the ashh2 mutant were calculated relative to wt levels with normalization to TUB8. Primers are given in Table IV.

### ChIP

Wt and ashh2-1 mutant plants were cultivated in growth chambers at 20° C. for 8 hrs of dark and 16 hrs of light (100 μE·m⁻²·s⁻¹). For each experiment 2-3 g of fifteen days old seedlings was crosslinked in 1% formaldehyde under vacuum until the tissue was translucent.

Chromatin immunoprecipitation was done as described in Gendrel et al (2005). The antibodies used for immunoprecipitation were anti-H3K9me2 (#07-212, Millipore), anti-H3K4me3 (#07-473, Upstate), anti-H3K36me2 (#07-369, Upstate) or anti-H3K36me3 (ab9050, Abcam). Immunoprecipitated chromatin was eluted in a total of 250 μl elution buffer (1% SDS, 0.1 M NaHCO₃) and after reversion of crosslinking, DNA was extracted using the Quiaquick PCR purification kit (Quiagen) and eluted in 100 μl elution buffer. 5 μl of a 4× dilution was used as a template for real-time PCR in a Lightcycler (Roche). Typically a program of: 1 cycle 95° C./10 min, 45 cycles of 95° C./20 s, 52° C./30 s and 72° C./30 s was used to amplify target sequences. The levels of H3K4me3, H3K9me2, H3K36me2 and me3 were estimated relative to input chromatin that was normalized to the level of methylation on the silent transposon Ta3 (Schmitz et al, 2009; Zhao et al, 2005), which is not affected by mutation in ashh2 (Grini et al, 2009). Two technical and two biological replicas were used for each antibody. Primers are given in Table IV.

### Expression and Purification of GST Fusion Proteins

For all GST fusion protein expression constructs the CW domains were cloned via Eco RI and Bam HI restriction sites into pSXG vector (Ragvin et al, J Mol Biol 337: 773-788 2004). The ASHH2 (nt 2547-2811) and VAL1 (nt 1575-1797) CW domains were cloned by PCR from Arabidopsis cDNA, MORC4 (nt 1236-1422) and ZCWPW1 (nt 714-942) CW domains were cloned from HEK293 cDNA.

Protein expression was performed in YT-G medium supplemented by 2 μM Zn acetate by incubation with 0.4 mM IPTG (Isopropyl β-D-1-thiogalactopyranoside) for 4 hours at 26° C. and purified by affinity chromatography using glutathione Sepharose as previously described (Ragvin et al, 2004).

Mutant versions of ASHH2 CW were generated by PCR using mutation-specific primers (Table IV) and subsequent annealing and primer extension to generate full-length, double-stranded mutant DNA. Mutant GST-CW proteins were cloned and expressed in pSXG as described above. All constructs were verified by DNA sequencing.

### Histone Peptide Binding Assays

Histone peptide binding assays were performed as described by Shi et al (2006) with biotinylated histone peptides from Upstate Biotechnology. The protein-peptide ratio used is indicated in the legends to the figures. Bound proteins were visualized by immunoblotting using rabbit anti-GST antibodies Z-5 (SC-456) from Santa Cruz at a 1:20,000 dilution, and a donkey anti-rabbit HRP conjugate (Amersham NA934) at a 1:10,000 dilution.

### Surface Plasmon Resonance

Surface plasmon resonance binding assays were performed on a BIAcore T100 biosensor according to the manufacturers protocols using immobilized, biotinylated H3 peptides monomethylated (0.54 ng), dimethylated (0.24 ng), or trimethylated (0.48 ng) on lysine 4. GST-tagged ASHH2 CW protein in five different concentrations in a range from 0.1 μM to 10 μM was injected for 2 min at a flow rate of 10 μl/min. Each sample injection was followed by injection of HBS-P buffer (10 mM HEPES pH 7; 150 mM NaCl) alone for 5 min at a flow rate of 5 μl/min. K_{d} values were obtained using the Biacore T100 Evaluation software 2.0.1. Measurements were repeated 2 to 5 times.

### NMR Spectroscopy

¹³C and ¹⁵N labelled ASHH2-CW was expressed and purified and subjected to NMR spectroscopy in the absence and presence of a histone H3K4me1 peptide.

Protein Expression.

¹³C and ¹⁵N labelled ASHH2 CW protein was expressed and prepared for NMR essentially as previously described (Rogne et al., 2008), briefly as follows: The cells were grown to an OD600 of 0.7 in unlabeled rich LB-medium at 37° C., harvested by centrifugation, washed and finally transferred into ¹⁵N/¹³C-labeled M9 defined medium (using [¹⁵N]NH₄Cl and [¹³C]glucose, Sigma). Cells from 4 L of LB-medium were transferred into 1 L of M9 medium and incubated for 1 hour to adapt the cells to the new medium and deplete the unlabeled metabolites. Protein expression was then induced by addition of IPTG to a final concentration of 1 mM. After 4 hrs, the cells were harvested by centrifugation, resuspended in TZNK buffer (50 mM TrisHCl pH8.5; 12 mM NaCl; 150 mM KCl; 100 μM ZnAcetate; 2 mM MgCl₂; 10 mM β-mercaptoethanol) and lysed using French press. The lysate was added Triton X-100 to 0.1% and incubated for 30 min on ice, before the lysate was cleared by centrifugation. The GST-ASHH2-CW fusion protein was affinity-purified on gluthatione Sepharose. The CW domain was cleaved from the GST moiety while one the beads by treatment over night at 4° C. with 1 U biotin-conjugated thrombin per mg fusion protein. Streptavidin Sepharose was added and Sterile Ultrafree-MC Centrifugal Filter Units (Millipore) were used to remove the Sepharose beads. The CW domain was concentrated using Amicon 10.000 NMWL centrifugal concentrators to a concentration of 13.76 mg/ml.

Synthetic H3 peptides (residues 1-16; ARTK(me1)QTARKSTGGKAY (SEQ ID NO: 30), ARTK(me2)QTARKSTGGKAY (SEQ ID NO: 30) and ARTK(me3)QTARKSTGGKAY) (SEQ ID NO: 30) were purchased from CASLO (Lyngby, Denmark).

NMR Sample Preparation.

The CW experiments were performed with samples containing 0.6-0.8 mM of CW and a ratio of histone peptides and CW of ˜1.1:1. The buffer was 20 mM sodium phosphate pH 6.5; 50 mM NaCl; 1 mM DTT; 10 μM ZnCl₂; and 0.2 mM DSS in H₂O:D₂O at a ratio of 95:5.

NMR Spectroscopy.

The following NMR experiments were performed to assign the backbone chemical shifts and determine structural restraints; 15N-HSQC, 13C HSQC, 15N NOESYHSQC, 13C NOESYHSQC, HNHA, HNCA, CBCAcoNH, CBCANH, HNCO, HNcaCO, HNcoCA, (H)CCcoNH, HcccoNH, HBHAcoNH, HBHANH HCCH-TOCSY (Davis et al. J. Magn. Reson. 98:207-216 (1992) 1992; Sattler et al., Progress in Nuclear Magnetic Resonance Spectroscopy 34:93-158 (1999) Journal of Biomolecular NMR, 23: 23-33 (2002) 1999). 1H-15N NOESY with a mixing time of 3s where performed to investigate the relaxation properties of the protein (Renner et al., 2002). All experiments were run on a 600 MHz Bruker Avance II spectrometer with four channels and a 5 mm TCI cryo probe at 25° C. ¹H-¹⁵N HSQC experiments were also obtained at 10° C., 15° C., and 20° C.

Data Processing.

Data were processed using Topspin 1.3 (Bruker Biospin). DSS was used as a chemical shift standard, and ¹³C and ¹⁵N data were referenced using frequency ratios as described by Wishart et al. J Biomol NMR 6:135-40 (1995) (1995).

Assignment.

For visualization, assignment the computer program SPARKY (Goddard et al., 2008) was used. The spectra were assigned using standard methods (Sattler et al., 1999, surpa). The chemical shifts are deposited with the Biological magnetic resonance data bank with accession code 17365.

Structure Calculation.

The ¹⁵N and ¹³C NOESY HSQC spectra were manually peak picked using SPARKY (Goddard et al., 2008, supra). NOESY upper distance constraints were generated by the CANDID rutine in CYANA 2.1. Torsion angle constraints were determined from the chemical shifts by the application of TALOS (Cornilescu et al., Journal of Biomolecular NMR 13:289-302 (1999) 1999). Temperature dependence more positive than −4.5 ppb/K for the amid proton was taken as a proof of the existence of a hydrogen bond (Baxter and Williamson, 1997, supra). J coupling constraints were determined for resolved peaks from the HNHA experiment using the procedure described by Vuister and Bax, J Am Chem Soc 115:7772-7777 (1993) (1993). Hydrogen bond restraints and Zn restraints was introduced in the final stage of structure determination. 100 structures were calculated using CYANA 2.1 and the 20 structures with the lowest energy were kept in the structure ensemble. The final structure ensemble is deposited in the protein data bank access code 217p. The final structure ensemble is deposited in the protein data bank accession code 217p and the chemical shifts have been deposited in the Biomagnetic resonance data bank with the accession code 17365.

Structures were rendered in MacPyMOL and surface potentials were calculated and displayed with PMV-1.5.4.

### Chromatin Pulldown

Chromatin pull-down (ChPD) was performed using crude chromatin in ChIP dilution buffer (1.1% Triton X-100, 1.2 mM EDTA, 16.7 mM Tris-HCL pH 8, 167 mM NaCl) prepared as for ChIP (Gendrel et al., 2005) incubated with 8 μg GST-CW fusion protein and control proteins (only GST alone, or together with GST-CW-W874A and GST-WIYLD) over night. For western blotting, the pulled down chromatin was washed three times in ChIP dilution buffer, run on a 15% SDS-PAGE and blotted onto a PVDF membrane. Blots were probed with the following antibodies: anti-H3 (ab1791; Abcam, 1:1000), anti-H3K4me1 (ab8895; Abcam, 1:1000), anti-H3K4me2 (07-030; Milipore, 1:1000), anti-H3K4me3 (ab8580; Abcam, 1:10000) and anti-H3K36me3 (ab9050; Abcam, 1:2000). For ChPD followed by qPCR on ASHH2 target genes, the complete ChIP protocol (Gendrel et al., Nat Meth 2: 213-218 2005) was used, exchanging antibodies with GST fusion proteins. Pulled down chromatin-CW complexes were eluted in a total of 250 μl elution buffer. The subsequent procedures were performed as for ChIP (see section above).

### Results

H3K36Me3 Methylation is Positively Correlated with Transcription of Tissue-Specific Genes

Although ASHH2 appears to be the enzyme responsible for global di- and trimethylation of H3K36, only a subset of genes are transcriptionally affected by ashh2 mutation. The effect of the ashh2 mutation on expression and histone marks was investigated for a selected panel of genes, with the aim of identifying features of the chromatin context in which ASHH2 is acting, assuming that the function of its CW domain is to render the enzyme sensitive to this chromatin context. With antibodies against H3K4me3, and H3K36me3, ChIP-analyses comparing wild type (wt) and ashh2 mutant seedlings were used on a set of tissue-specific genes with differential expression profiles in seedlings and flowers: 1) APETALA1 (AP1), MYB99, and the transcription factor NAC25 predominantly expressed in the inflorescences; and 2) DISRUPTION OF MEIOTIC CONTROL 1 (AtDMC1), MADS-BOX AFFECTING FLOWERING 1 (MAF1) and FLC with low and tissue-specific expression both in vegetative and reproductive tissues. It was previously shown that AP1, MYB99, NAC25 and AtDMC1 are down-regulated in ashh2 mutant inflorescences and associated with mutant phenotypes (Grini et al, 2009). MAF1, which similar to FLC is involved in determination of flowering time, is transcriptionally down-regulated both in ashh2 seedlings and inflorescences (Grini et al, 2009, supra; Kim et al, 2005 Plant Cell 17 3301-3310; Xu et al, 2008, supra; Zhao et al, Nat Cell Biol 7: 1156-1160 2005). ACTIN2 and GAPA (GLYCERALDEHYDE 3-PHOSPHATE DEHYDROGENASE A SUBUNIT), which show high expression but little tissue specificity, were also included in the ChIP analysis.

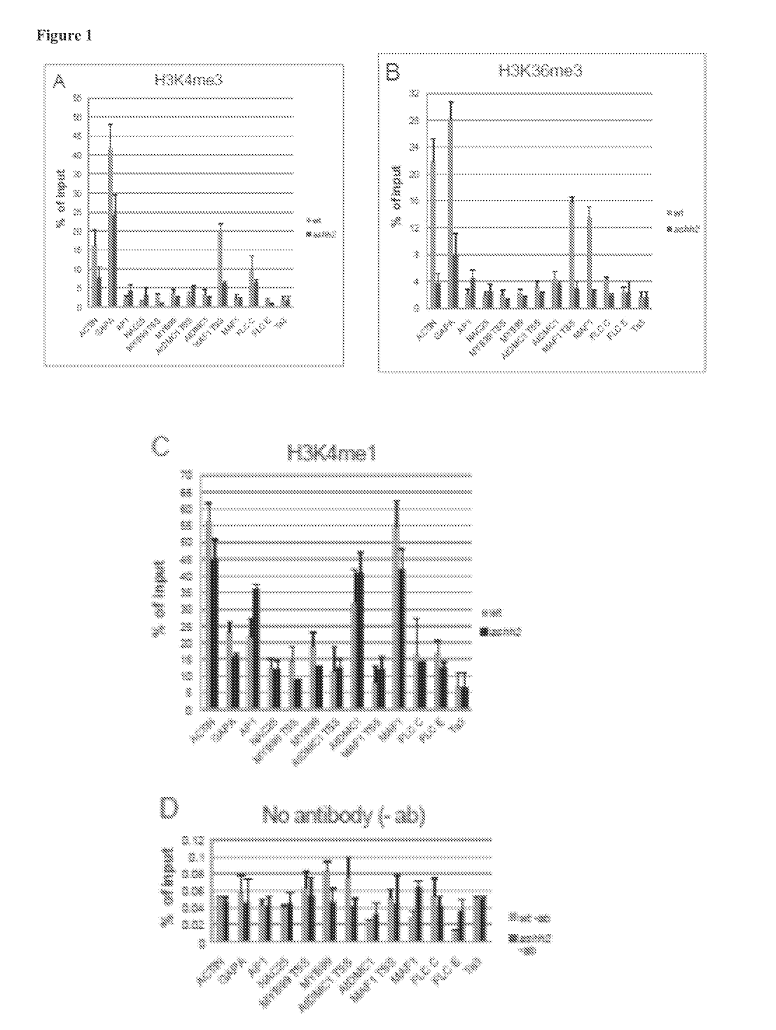

H3K4me3 levels were high in the wt for these two strongly expressed genes, and around the transcriptional start site (TSS) of MAF1 and the beginning of first intron of FLC. In the ashh2 mutant, the levels of H3K4me3 were reduced (FIG. 1A). The other genes tested were largely unaffected in the ashh2 mutant seedlings. For AP1, MYB99, and AtDMC1, significant reductions of H3K36me3 levels that correlated with reduced transcription, has been demonstrated in ashh2 mutant inflorescences (Grini et al, 2009). These genes are not active in seedlings, and had the lowest K36me3 levels in this tissue, with similar levels in wt and the mutant. This was also the case for AtDMC1 and a downstream region of FLC (FIG. 1B). MAF1, which is substantially downregulated both in ashh2 seedlings and inflorescences (Grini et al, 2009; Xu et al, 2008), displayed a reduction of K36me3 in the mutant. A reduction of H3K36me3 level was also seen in the first intron of FLC as previously reported (Xu et al, 2008) (FIG. 1C). ACTIN2 and GAPA exhibited significant reductions in H3K36me3 levels in the mutant seedling samples (FIG. 1C).

These data indicate that the level of H3K36me3 methylation reflect the level of expression of a gene, but also that the H3K36me3 mark is not needed for sustained expression of genes with a high, constitutive expression level, like ACTIN2 and GAPA.

Global ChIP Data Indicates that ASHH2 has a Bias for Tissue-Specific Genes

To investigate whether the genes with ASHH2-dependent regulation had particular characteristics, genes from a microarray experiment (GSE22990 at the GEO database) as well as genes up- and downregulated in previously published experiments with ashh2 mutant seedlings (ccr1 and sdg8 (Cazzonelli et al, 2009, supra; Xu et al, 2008, supra); see Materials and methods) were surveyed for the presence of H3K4me3, H3K27me3 and H3K36me2 (Table II), using published global ChIP data covering 24468 genes (Oh et al, 2008). Over 84% of the down-regulated genes had K4me3 marks, either alone or in combination with other marks, indicating that ASHH2 preferentially associates with transcribed genes, known to be enriched in this mark around the transcription start site. Consistent with this, genes with H3K27me3 marks only, which are likely to be silent, were significantly underrepresented. Genes with all three marks, likely to be tissue-specific or developmentally regulated (Oh et al, 2008), were most significantly overrepresented amongst the downregulated genes (Table II). None of these biases were found amongst the up-regulated genes, of which only two genes were common to the three microarray sets, indicating that the observed upregulation is a secondary effect of the ashh2 mutations. The 45 downregulated genes found common to two or three of the microarray datasets, of which nine encode transcription factors, are more likely to be direct targets of ASHH2. Of these genes, 15.7% have triple marks, compared to 2.5% in the global gene set. FLC, known as a target of ASHH2, is among them.

TABLE II
Representation of ashh2 downregulated genes with different
chromatin methyl marks.

| | Global^{a} | | ashh2-1^{b} | | | sdgS-2^{b} | | | ccr1^{b} | | |
|---|---|---|---|---|---|---|---|---|---|---|---|
| Chromatin methyl marks | N | % | N | % | <P | N | % | <P | N | % | <P |
| H3K4me3 | 2929 | 12.0 | 33 | 24.4 | 2 × 10⁻⁵ | 7 | 8.3 | ns | 12 | 12.9 | ns |
| H3K4me3 + H3K36me2 | 12012 | 49.1 | 60 | 44.4 | ns | 46 | 54.8 | ns | 53 | 57.0 | ns |
| H3K36me2 | 1671 | 6.8 | 2 | [symbol] | 0.02 | 1 | [symbol] | 0.05 | 1 | [symbol] | 0.05 |
| H3K4me3 + H3K27me3 | 1245 | 5.1 | 11 | 8.1 | ns | 8 | 9.5 | ns | 3 | 3.2 | ns |
| H3K27me3 | 5724 | 23.4 | 14 | [symbol] | 0.002 | 11 | [symbol] | 0.05 | 7 | [symbol] | 0.005 |
| H3K36me2 + H3K27me3 | 280 | 1.1 | 0 | 0.0 | ns | 1 | 1.2 | ns | 0 | 0.0 | ns |
| H3K4me3 + H3K27me3 + | 607 | 2.5 | 15 | 11.1 | 5 × 10⁻⁶ | 10 | 11.9 | 10⁻⁶ | 17 | 18.3 | 5 × 10⁻⁶ |
| H3K36me2 | | | | | | | | | | | |
| Total number of genes | 24468 | | 135 | | | 84 | | | 93 | | |
| H3K4me3 in total | 16793 | 68.6 | 119 | 88.1 | 0.001 | 71 | 84.5 | ns | 85 | 91.4 | 0.001 |
| H3K27me3 in total | 14570 | 59.5 | 77 | 57.0 | ns | 58 | 69.0 | ns | 71 | 76.3 | 0.05 |
| H3K36me2 in total | 7856 | 32.1 | 40 | 29.6 | ns | 30 | 35.7 | ns | 27 | 29.0 | ns |

^{a}Chromatin enrichment groups and number of genes in each group according to Oh et al. (2008). In the three bottom rows the number of genes is given for each mark, irrespective of to presence of other marks or not.
^{b}Chromatin enrichment according to Oh et al (2008) for genes down-regulated in three independent microarray experiments on ashh2 mutants seedlings; ashh2-1 - present study, sdgS-2 (Xu et al., 2008) and ccr1 (Cazzonelli et al., 2009). Values significantly higher percentage than for the whole genome is shown in boldface, while values significantly lower than for the whole genome are shown in italicised boldface. N = number of genes. ns—not significant.

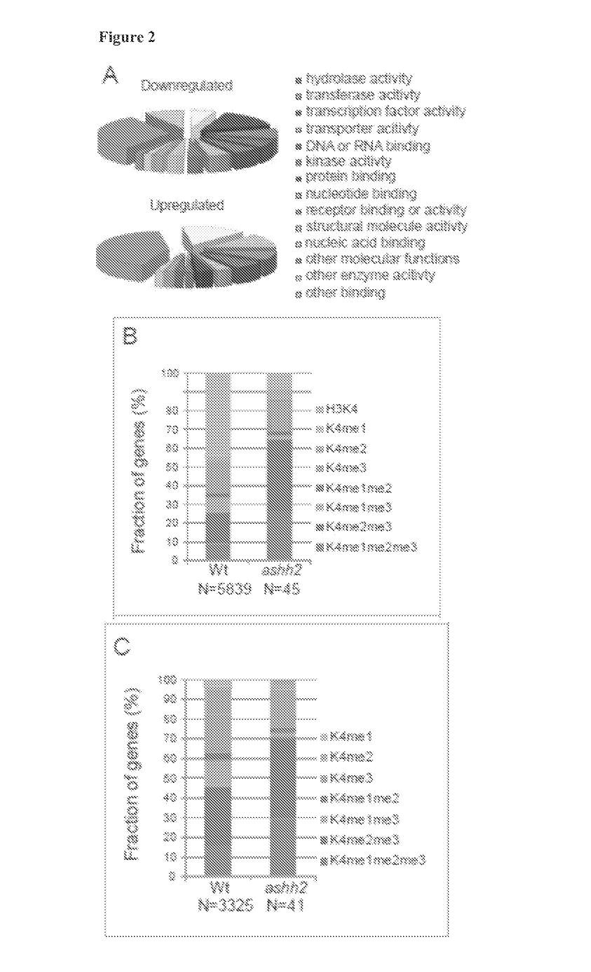

The 45 genes downregulated in ashh2 mutants and the panel of genes investigated in the ChIP experiment were also surveyed for H3K4me1, me2, me3, and H3K27me3 using a published, global dataset for Arabidopsis seedlings (Zhang et al, 2009, supra) (Table III; FIGS. 2B and C). The inflorescence-specific genes AP1, NAC25 and MYB99, which are silent in seedlings, were only marked by the repressive K27me3. The global data show that genes marked by K4me1/me3, K4me1/me2/me3 and K4me3 are highly expressed and exhibit very low levels of tissue specificity, whereas K4me1, K4me2 and K4me1/me2 genes are expressed at very low levels, and are highly tissue-specific (Zhang et al, 2009). Accordingly, the two highly and generally expressed genes, ACTIN2 and GAPA, used in ChIP experiments (FIG. 1), have K4me1/me2/me3 and K4me1/me3 marks, respectively, while the low-expressed, tissue-specific AtDMC1 and MAF1 are marked with K4me1/me2 and K4me1, respectively. Consistent with this, the ChIP results showed that there are much higher levels of K4me3 on ACTIN2 and GAPA than the other genes tested (FIG. 1A).

According to the dataset of Zhang et al. (2009), surpa 43% of 5839 genes investigated in detail were devoid of H3K4me marks (FIG. 2B), while this category was significantly underrepresented among the 45 ASHH2-dependent genes (8.9%; FIG. 2B, Table III). The fraction of genes with the K4me1 mark was similar to wild type (31.1 versus 32%), while K4me2 and K4me3 were overrepresented (77.1 and 86.7% versus 38.8% and 55.4%, respectively). When considering combinations of K4me marks, K4me2/me3 and K4me1/me2/me3 were overrepresented among genes downregulated in the ashh2 mutant seedlings (FIGS. 2B and C).

Again this supports that ASHH2 is associated with transcribed genes, and furthermore that ASHH2 has a particular preference for transcribed genes with K4me2 and K4me1/me2 marks.

### The ASHH2 CW Domain Binds Methylated Lysine 4 on H3

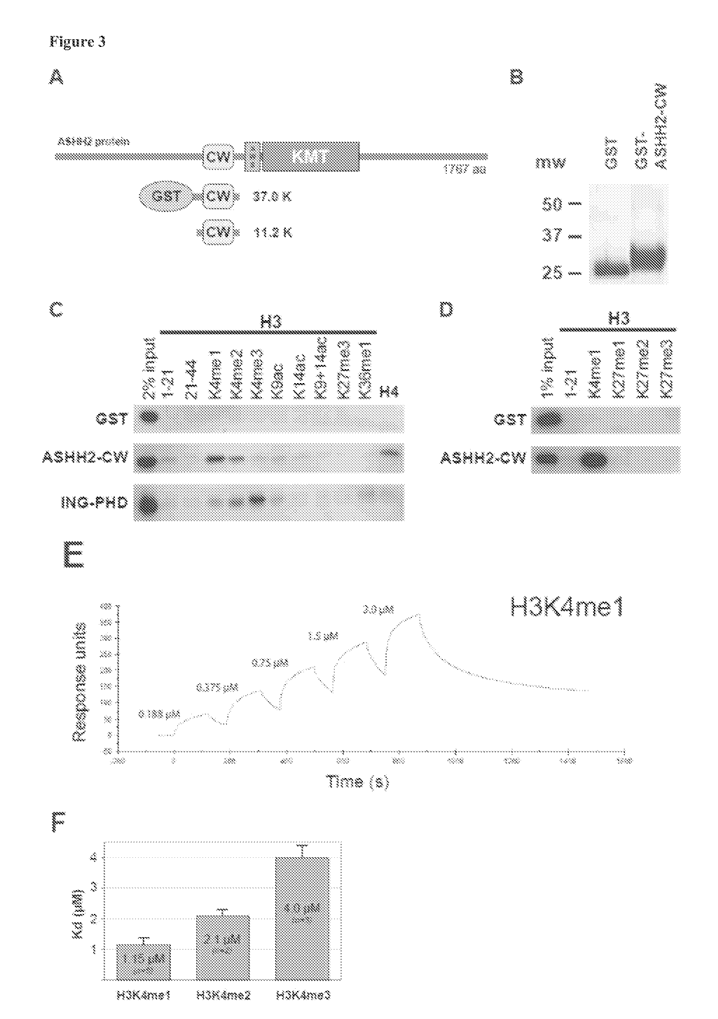

The occurrence and proximity of the CW domain to the SET domain in ASHH2 (FIG. 3A), is reminiscent to many other histone modifying enzymes and prompted an investigation of whether CW is a histone recognition module that explains the association of ASHH2 with transcribed genes and methylated H3K4 as identified above. The ASHH2 CW domain was expressed and purified as a GST-fusion protein from bacteria (GST-CW_{ASHH2}) and tested for binding to a panel of immobilised histone tail peptides (FIGS. 3A and B). As shown in FIG. 3C the ASHH2 CW domain can bind histone tail peptides with comparable avidity as the ING2 PHD finger (Shi et al, Nature 442: 96-99 2006). Unlike the ING2 PHD finger, however, ASHH2 CW shows preference for mono- and dimethylated H3K4 peptides, while its binding to the H3K4me3 is close to background levels. The preference for mono- and dimethylated H3K4 peptides was quantified by surface plasmon resonance using BIAcore (FIGS. 3E and F). The K_{d}s for H3K4me1 and H3K4me2 were 1.15 μM and 2.1 μM, respectively, comparable to the affinities of ING2 PHD finger to the H3K4me3 peptide (Pena et al, 2006). K_{d} for binding to H3K4me3 in these experiments was 4 μM.

From the data in FIG. 3C, it is also evident that the ASHH2 CW domain is sequence selective, as it only binds the methylated H3K4 peptides, and not the trimethylated H3K27 and H3K36 peptides, nor the other peptides included on the panel. The preference of ASHH2 CW for mono- and dimethylated H3K4 peptides is shared with MBT domains (Bonasio et al, 2010). The MBT domains show little sequence selectivity, however, as they bind several mono- and dimethylated lysines on both H3 and H4 peptides. In contrast, the ASHH2 CW domain is specific for methylated H3K4 peptides, as no binding was observed for any of the three methylated forms of H3K27-peptides (FIG. 3D). This indicates that the CW domain may have a more extensive interaction with the histone H3 tail residues near K4.

### Binding to Methylated Histone Tails is a Shared Feature Among CW Domains

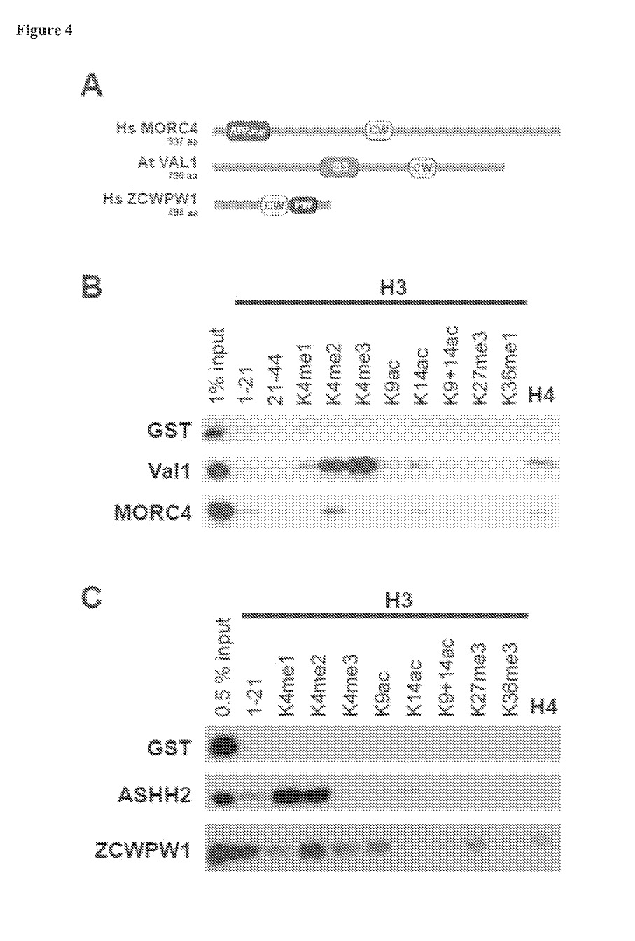

In order to determine if histone tail binding a general feature of CW domains several other CW domains were assayed in the histone tail binding assay. As is evident from FIG. 4, the CW domains of human MORC4 and ZCWPW1 and Arabidopsis VAL1 all bind H3 peptides methylated on lysine 4. While the MORC4 CW domain shows a preference for H3K4me2, the CW domains of ZCWPW1 and VAL1 show a clear preference for H3 Kme2 and me3 peptides. Based on these data, most, if not all, CW domains are H3K4-selective recognition modules, but their preference for the methylation states may vary. Furthermore, as the different methylated forms of H3K4 are associated with chromatin of active or poised genes and/or their enhancers, it is contemplated that CW proteins are acting on active or poised chromatin.

### Solution Structure of the ASHH2 CW Domain

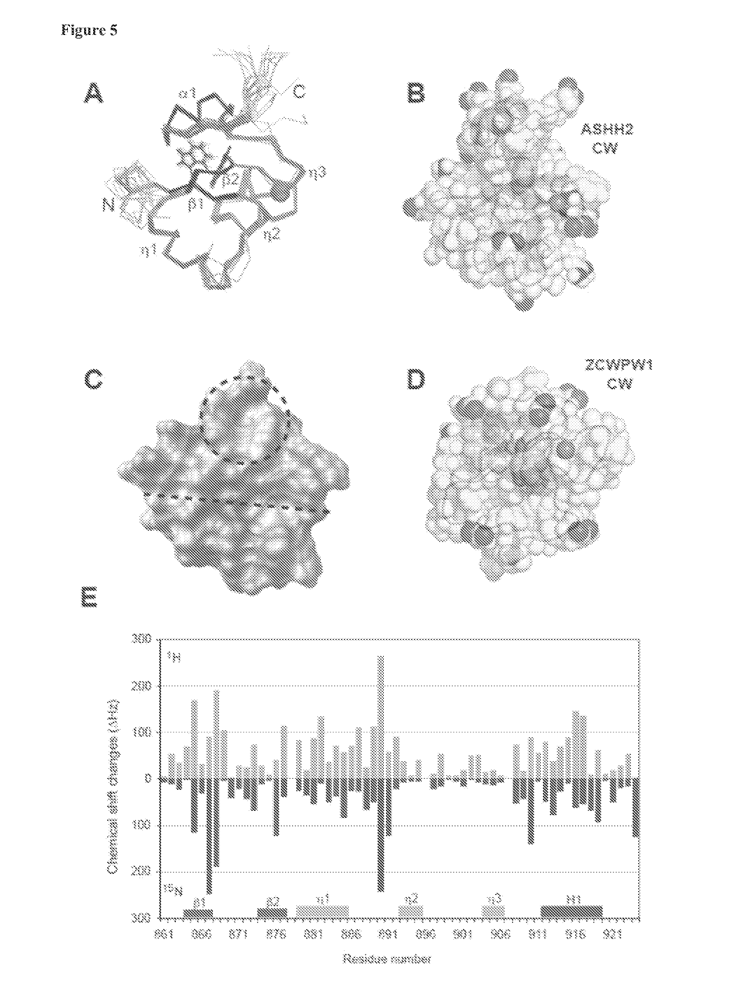

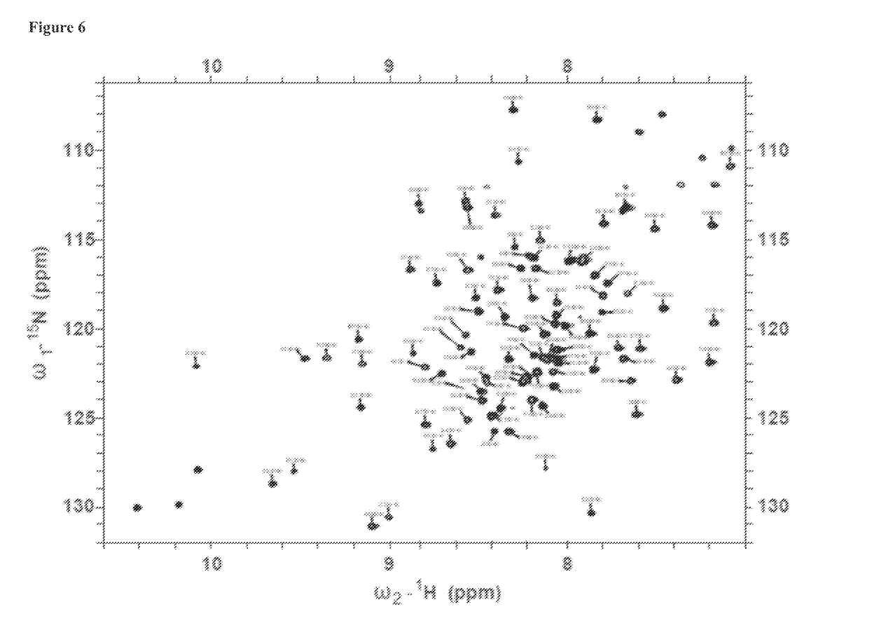

To further investigate the structure of the CW domain and its mode of interaction with the histone tail, the solution structure of the ASHH2 CW domain was solved using NMR spectrometry (FIG. 5A-C, FIG. 6). The HSQC spectrum indicated that the molecule was structured and the heteronuclear 3D NMR spectra were assigned using standard methods. The chemical shifts of the cysteine residues are all in agreement with metal-bond cysteines, Cys868, 871, 893, and 904, while Cys931 is non oxidized. It was thus concluded that the former four cysteines are bound to Zn²⁺.

Comparison of this structure to the reported structure of the human ZCWPW1 CW domain (pdb:2e61; FIG. 5D) revealed that both domains share a common structural core consisting of a two-stranded β-sheet and a Zn²⁺-coordinating quartet of cysteines. Both structures also show three short helical elements (η1-3), although their extents are variable within the ensemble of models for ASHH2 CW. Inspection of the molecular surface (FIG. 5C) revealed a conspicuous cleft is running across the front surface with a shallow pocket lined by two of the conserved tryptophans (W256 and W267 in ZCWPW1; W865 and W874 in ASHH2). The solution structure of ZCWPW1 CW in complex with a H3K4me3 peptide has been reported (He et al, 2010). This structure shows that the histone peptide traverses the cleft, and the methylated ε-amino group of lysine 4 is bound in the pocket above, which serves as an aromatic cage. It is contemplated that the H3K4me1-peptide could bind the core of the ASHH2 CW domain in a similar fashion.

In the ASHH2 CW structure, an extended α-helix (α1) is formed by residues 912-919, followed by a less structured C-terminal segment (FIG. 5A). This helix has a strong amphipathic character and it is situated on top of the pocket with its hydrophobic side facing the core surface and occluding the aromatic cage (FIG. 5B). This helix is not part of the conserved core of CW domains and different families of CW domains have different C-terminal extensions. In ZCWPW1 CW, the C-terminal extension provides a third tryptophan to the aromatic cage (He et al, 2010).

### NMR Spectroscopy Data Supports the Predicted Histone Tail Binding Site on CW

To investigate the interaction of the histone peptide with the ASHH2 CW domain, the chemical shift values of the domain in the absence and presence of a monomethylated H3K4 peptide were determined by NMR (FIG. 6). The HSQC spectrum showed that the domain is structured, both in the presence (FIG. 6) and absence of histone peptide. Upon histone peptide binding, a number of discrete chemical shift changes were observed (FIG. 5E). The most prominent shifts occurred around R867 and R890, which are juxtaposed on each side of the cleft. There were also moderate shifts in the central, variable region (which forms lower part of the cleft), indicating that this part of the structure is changing conformation upon histone tail binding. The residues near the zinc-coordinating cysteines showed less changes, in agreement with their structural role in the core of the domain. Only one of the two conserved tryptophans in the predicted methyllysine-binding pocket showed a significant change, namely W865. There was also a significant change for the adjacent R876 which is positioned in the outer end of the cleft. The C-terminal extension, including helix α1, also showed significant changes, indicating that this part of the molecule is moving when the histone peptide binds. The present invention is not limited to a particular mechanism. Indeed, an understanding of the mechanism is not necessary to practice the present invention. Nonetheless, it is contemplated that a movement of the C-terminal helix away from the aromatic cage is taking place as the H3K4me1 peptide binds. In summary, these NMR data confirm binding of the histone tail to the CW domain and the chemical shift changes are in good agreement with a mode of binding involving the aromatic cage.

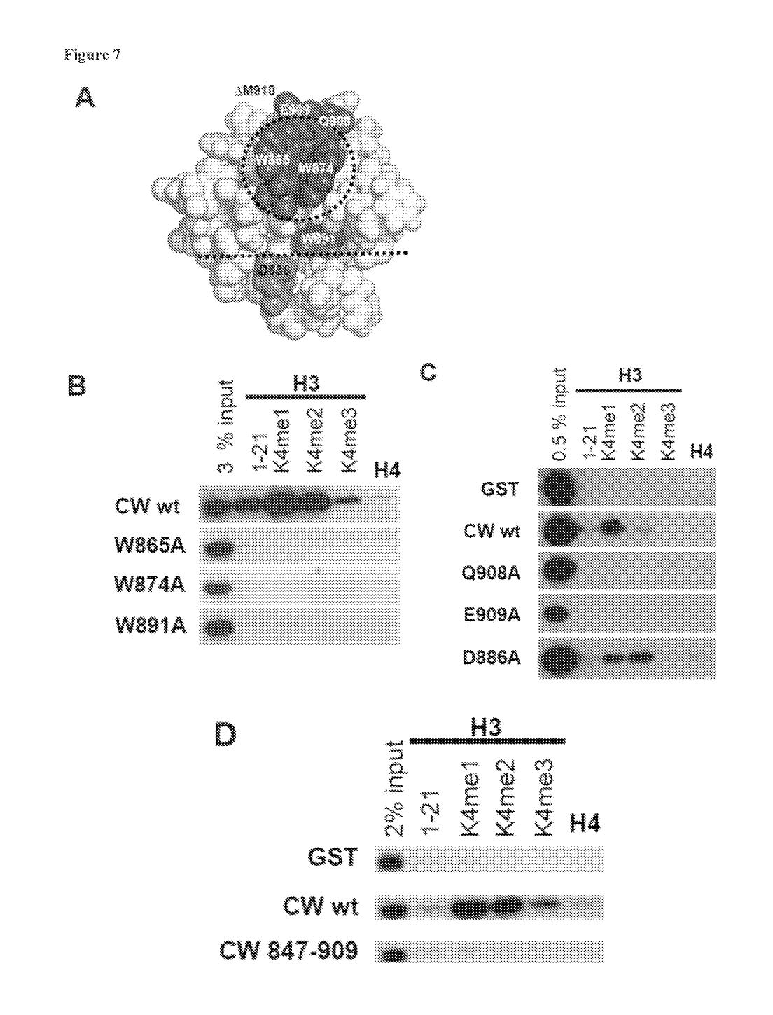

To further corroborate these data, several point mutations in and around the putative histone tail binding site were generated and tested for binding (FIG. 7A). Mutation of the three tryptophans 865, 874 and 891 to alanine abolished histone tail binding (FIG. 7B). Two of these (W865 and W874) form the predicted methyllysine binding site while W891 is located in the presumptive histone tail binding cleft. Mutation of the two residues Q908 and E909, positioned above the putative methyllysine binding pocket also abolished binding (FIG. 7C). According to a model for histone tail binding, these two residues may contribute to polar or ionic interactions with the positively charged E-amino group of K4. Mutation of the non-conserved D886 to alanine positioned in the lower part of the cleft resulted in relaxed specificity with strong binding also for K4me2 (FIG. 7C).

The C-terminal truncation of the domain from residue M910 resulted in loss of binding (FIG. 7D) indicating that the C-terminal extension, including helix α1, plays an important role in histone peptide binding.

### The ASHH2 CW Domain Binds Nucleosomal Histones

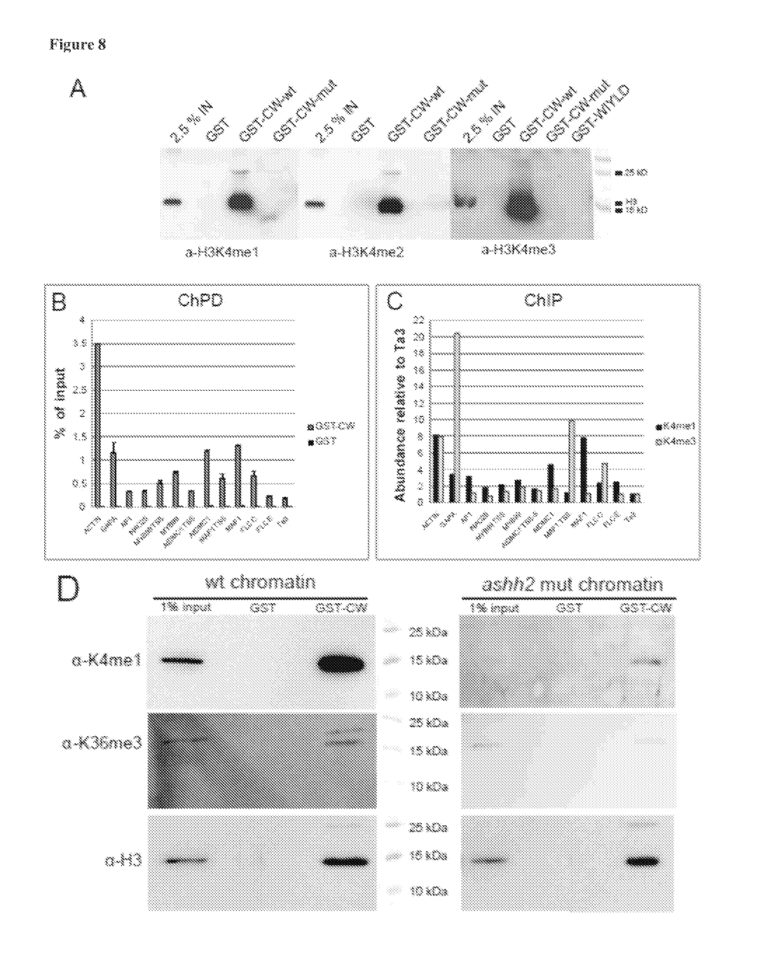

To investigate whether the CW domain can bind histone H3K4me tails in a nucleosomal context, the GST-ASHH2-CW fusion protein was used in pulldown experiments with chromatin prepared from Arabidopsis seedlings. Strong signals were detected with antibodies against each of the three H3K4 methylation states (FIG. 8A). No band could be detected when using the non-binding mutant version of the CW domain (W874A), consistent with the peptide binding data. In contrast, the WIYLD domain of the Arabidopsis SET-domain protein SUVR4 (Thorstensen et al, Nucl Acids Res 34: 5461-54702006), included as a negative control, did not pull down H3K4 methylated chromatin, indicating that the binding is specific for the CW domain. Quantification of the bound chromatin relative to input chromatin, indicated that CW binds H3K4me1 in Arabidopsis chromatin somewhat stronger than H3K4me2 (4.4 versus 3.3 times of input material (2.5%)) as compared to 2.1 times for H3K4me3. It should be noted that, (1) the chromatin in these experiments is most likely a mixture of mono-, di-, and tri-nucleosomes, and (2) according to the analysis of chromatin marks of putative ASHH2 target genes, H3K4me1, H3K4me2 and H3K4me3 often co-reside (FIGS. 2B and C), and that the genes which in the ChIP experiment show significant reduction in H3K36me3 (FIG. 1B) were expressed genes with additional H3K4me1/me2 or H3K4me1 marks.

To investigate whether the CW domain targets genes regulated by ASHH2, DNA from seedling chromatin pulled down by the GST-CW_{ASHH2} fusion protein was analyzed by real-time PCR. This chromatin pull-down (ChPD) experiment demonstrated that CW binds chromatin associated with these genes significantly above background levels (FIG. 8B). FLC, proven to be targeted by ASHH2 in vivo (Ko et al, EMBO J. 29:3208-3215 2010), was detected in the CW-bound chromatin, thus demonstrating the ability of the CW pull down to identify in vivo targets of ASHH2. The CW domain most strongly pulled down chromatin associated with genes which show substantial reduction in H3K36me3 levels in the ashh2 mutant (ACTIN2, GAPA, MAF1 and FLC), indicating that the CW domain may contribute the targeting of ASHH2 to chromatin associated with these genes. For MYB99, AtDMC1 and MAF1, recovery was higher with primers downstream of the TSS. When comparing the CW_{ASHH2} ChPD (FIG. 8B) and the ChIP experiments (FIG. 8C), it is evident that the recovery profiles are very similar for ChPD and H3K4me1, and also for K36me3. Only for the very H3K4me3-rich GAPA gene and the transcriptional start site of MAF1, the H3K36me3 levels were more similar to H3K4me3 than to K4me1.

The panel of tested genes showed no dramatic difference in H3K4me1 levels between wt and the ashh2 mutant (FIG. 1C). Western blots of ChPD experiments show that GST-CW_{ASHH2} efficiently pulled down H3K4me1 marked chromatin of wt seedlings (FIG. 8D, upper compared to the H3 control in lower panel). Antibodies against H3K36me3 revealed the presence of this mark on the chromatin pulled down by GST-CW_{ASHH2} (FIG. 8D, middle panel), indicating that H3K4me1 and H3K36me3 co-reside on the same or neighbouring nucleosomes. If H3K36me3 was deposited on chromatin independently of H3K4me1, one would expect that GST-CW_{ASHH2} pulled down H3K36me3 chromatin equally well in the mutant compared to the wt, relative to input. However, less H3K36me3 chromatin was pulled down from ashh2 chromatin than from wild type chromatin relative to input (FIG. 8D, middle panel). This indicates that the chromatin regions pulled down by CW was highly affected by the ashh2 mutation. Together with the finding that CW pulls down chromatin from genes with the most significant reduction of H3K36me3 in the mutant (FIGS. 8B and C), these data indicate that the H3K36me3 mark, mediated by ASHH2 activity, is closely associated with H3K4me1-marked chromatin bound by the CW domain.

The CW Domain is a New Histone Recognition Module with Specificity for Methylated H3K4

It is described here the CW domain as a new histone recognition module with specificity for histone H3 tails methylated on lysine 4. For the ASHH2CW the histone tail binding was demonstrated in four different ways, by: (1) histone tail-binding pull-down assays; (2) surface plasmon resonance; (3) nucleosome binding assay; and (4) by NMR. The affinity for the mono- and dimethyl H3K4 peptides are in the micromolar range, comparable to PHD fingers and other histone recognition modules. Histone tail binding was also demonstrated for three additional CW domains, indicating that this is the generic molecular function of CW domains. The ZCWPW1 CW domain has recently been to bind H3K4me3, however, under conditions utilized herein it also binds H3K4me2 (He et al, Structure 18: 1127-1139 2010).

Among the families of H3K4-specific recognition modules, CW has a novel profile of ligand selectivity, with members showing preference for either me1 and me2 (ASHH2), me2 and me3 (VAL1 and ZCWPW1), or me2 (MORC4). This is distinct from e.g. PHD fingers, which bind either trimethylated or unmethylated H3K4 peptide; the trimethyllysine-specific double tudor domain and double chromo domains; and the MBT domains, which also bind mono- and di-methylated lysines, but in several different sequence contexts (Bonasio et al, 2010).

A notable feature of CW domains concerns their phylogenetic profile. They are found in plants and chordates as well as certain protist lineages and the cnidarian Nematostella vectensis (see Pfam:zn-CW, PF07496, for details). One is therefore led to speculate that CW proteins were lost in lineages such as insects and nematodes. Even more remarkable is the fact that none of the mammalian and plant CW proteins are orthologous,—yet, they are involved in phenomena related to chromatin and gene regulation (Table I). Since CW domains in both kingdoms have very similar molecular functions (recognition of methylated H4K4-tails), it is contemplated that CW proteins allow plants and chordates to employ these histone marks in different ways. Four of the CW proteins in Arabidopsis have methyl-CpG binding domains (see Table I).

Unlike PHD fingers, bromodomains, chromodomains, and several other histone recognition modules, CW domains only occur in one copy per protein and they rarely co-occur with other histone recognition modules.

Structure of the CW Domain and its Mode of Interaction with Histones

Comparing the structures of the CW domain of ASHH2 and that recently reported for ZCWPW1 (He et al, 2010) reveals that both domains share a common structural core built around two β-strands and a Zn²⁺-binding site, reminiscent of PHD fingers. A cleft traverses one side of the domain, just underneath a pocket containing two conserved tryptophans forming an aromatic cage reminiscent of the aromatic cages of other methyllysine-binding domains, as on the PHD fingers, the chromodomains and in the bottom of the cavities in the MBT domains (Taverna et al, 2007, supra). In ZCWPW1 CW, this is the binding site for the methylated H3 peptide (He et al, 2010). It is contemplated that these conserved features form the binding site on ASHH2 CW. This is supported by both NMR spectrometry and site-directed mutagenesis (FIGS. 5 and 7).

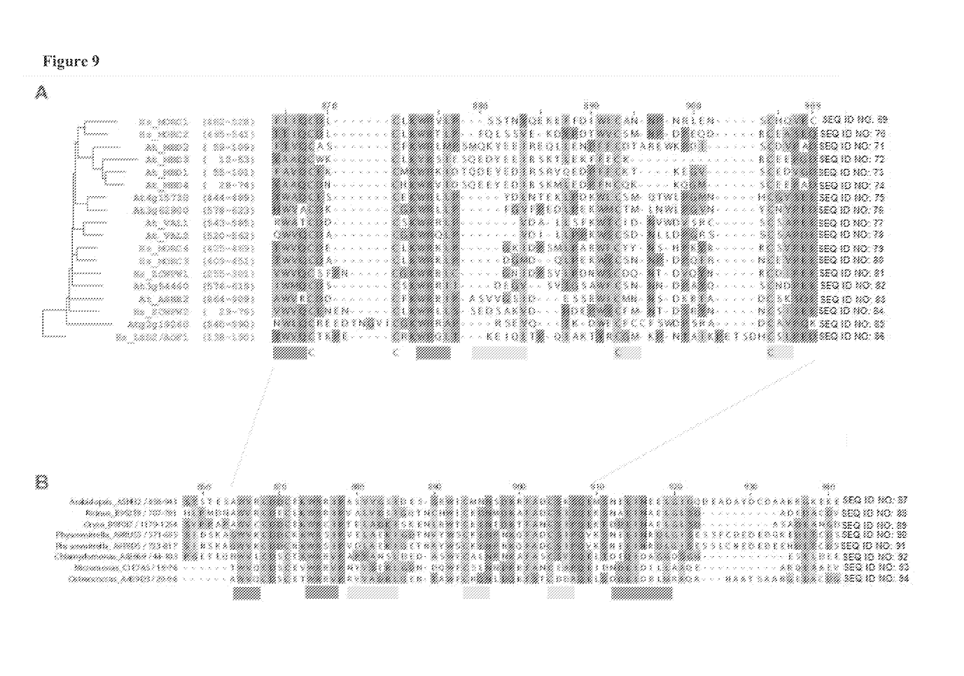

For ZCWPW1 CW it was also shown that the N-terminal amino group of A1 of the histone tail is interacting with an aspartate carbonyl oxygen. In ASHH2 CW, D869 is placed in an equivalent position. The lower part of the cleft, which interacts with the histone peptide shows sequence variation (FIG. 9; residues 870-880 in ASHH2). One explanation for this variation could be that some CW domains also recognise methylated lysines on non-histone proteins. Another possibility is that the CW domains are differentially sensitive to other modifications on the histone tail (e.g, R2, T3, and T6).

The most remarkable feature of the two CW domain structures is, however, the role of the non-conserved C-terminal extension for H3K4me-binding. Each subfamily of CW domains have a unique C-terminal extension, presenting a third tryptophan for the aromatic cage in ZCWPW1 while an amphipathic helix in ASHH2. Given the observations that different CW domains show different preferences for the three states of K4-methylation, it is contemplated that the family-specific C-terminal embellishments serves as determinants for recognition of the differentially methylated H3K4, a novel feature among histone recognition modules.

ASHH2 Activity Correlates with Transcriptional Output of Tissue-Specific and Developmentally Regulated Genes

The importance of the K4me reading function of the CW domain is indicated by the underrepresentation of genes without K4me marks among putative ASHH2 target genes. Furthermore, the inflorescence-specific genes AP1, MYB99 and NAC25 are not affected by ashh2 in seedlings where they are silent and only marked with H3K27me3. For the tissue-specific genes tested by ChIP there is a correspondence between transcriptional activity, H3K36me3 marks and ASHH2 activity, as mutation in ashh2 leads to a reduction both in transcript levels and H3K36me3 levels. Using global ChIP data, the chromatin marks of a larger number of genes affected by mutation in ashh2 were surveyed. This analysis showed a significant overrepresentation of H3K4me3/H3K36me2/H3K27me3 triple-marked genes (Table II), which indicates tissue-specificity or developmental regulation, with H3K27me3 associated with silent genes in cells where the gene is not expressed (Oh et al, PLoS Genet. 4: e1000077 2008). Alternatively the three marks may reside on the same chromatin as a specific means of controlling expression of genes involved in differentiation. FLC is such an ASHH2-regulated gene with triple marks (Pien et al, 2008; Schmitz et al, 2009; Xu et al, 2008). Interestingly, genes encoding transcription factors, many of which are tissue-specific, are overrepresented among genes depending on ASHH2 for maintenance of transcription levels in seedlings. A substantial number of transcription factors were also found downregulated in ashh2 inflorescences (Grini et al, 2009, supra).

The CW Domain May Contribute to ASHH2's Preference for Genes with H3K4 Methylation

In Arabidopsis H3K4 methylation generally localizes to the promoters and transcribed regions of genes (Zhang et al, 2009, supra). H3K4me3 is in particular associated with transcribed genes, and H3K4me2 often co-occurs with H3K4me3 in the 5′-end of genic regions, while H3K36me2 increases towards the 3′ end (Oh et al, 2008). H3K4me1 on the other hand, is found in internal regions especially in long genes (>4 kb) and is correlated with CpG DNA methylation in transcribed regions (Zhang et al, 2009 supra).

The experiments described herein indicate that ASHH2 has a strong preference for H3K4 methylated genes, especially those with combinations of K4 methylation states, for instance the combination K4me2me3 (Table III) associated with moderate expression levels and moderate tissue-specificity (Zhang et al, 2009 supra). ChPD indicated a reduction in H3K36me3 chromatin pulled down with the CW domain from the ashh2 mutant, compared to the total ashh2 chromatin. This indicates that H3K36me3 is generally associated with H3K4me1 which is the preferred target for ASHH2 CW. qPCR with DNA from ChPD by the CW domain showed that CW interacts with the target genes used in the study, including FLC, which has also been shown to bind ASHH2 in ChIP (Ko et al. 2010, supra). The lowest qPCR levels were found for the genes that are not affected by mutation in ASHH2 with respect to transcription levels and chromatin marks. Furthermore, the profile of abundance of the putative target genes and FLC is similar with ChPD, H3K4me1 and HeK36me3 ChIP experiments.

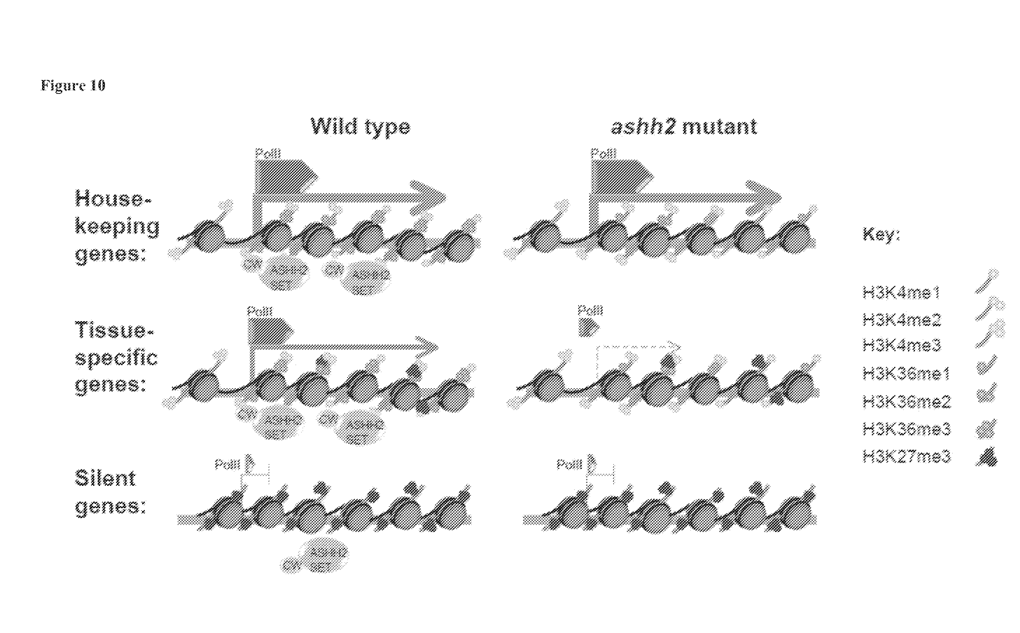

Therefore, a model for ASHH2 function is that the CW domain first positions the protein near the transcription start site by binding to H3K4me2 (and/or weakly to H3K4me3), and that binding to K4me2, and in longer genes K4me1 along the body of the gene, is accompanied with H3K36me3 methylation (FIG. 10). H3K4me2 often co-resides with the repressive mark H3K27me3 (Zhang et al, 2009, supra), and K36me3 marks are needed to maintain expression if such repressive marks are present. For genes with high expression levels and devoid of repressing marks, this maintenance function may not be needed. Therefore, reduction in H3K36me3 marks does not affect the transcription level of highly expressed genes like ACTIN2 or GAPA.

### The Different Specificities of the CW Domains Contribute Either to Maintenance or Changes In Gene Expression and Chromatin Status

Different CW domains show preference for different methylation states of H3K4. Reading of K4me1 and me2 may direct ASHH2 HMTase activity to transcribed genes, which could contribute to sustained gene expression (FIG. 10). VAL1 on the other hand, a transcriptional repressor of the related ABI3/FUS3/LEC2 (AFL) transcription factors controlling the maturation program of Arabidopsis seed development (Aichinger et al, PLoS Genet. 5: e1000605 2009), has strongest preference for K4me3 found at transcription start sites. Following germination, VAL1 is involved in repressing the embryonic pathway so that normal leaves can form (Guerriero et al, New Phytologist 184: 552-565 2009). It has been suggested that the VAL proteins recruit histone deacetylases and chromatin remodeling factors to repress the AFL genes (Suzuki & McCarty, Plant Physiol 143: 902-911 2008; Tanaka et al, Gene 397: 161-168 2007): Thus, in the case of VAL1 the CW domain may, in contrast to CW in ASHH2, contribute to a switch from an expressed to a repressed chromatin status.

TABLE III
Genes downregulated in ASHH2 mutant seedlings and their epigenetic marks

| | | | GO | |
|---|---|---|---|---|
| Gene ID^{a} | Oh et al 2008 | Zhang et al 2009^{b} | term^{c} | Encoded protein |
| At1g23960 | H3K4me3 | H3K4me1me2me3 | | Expressed protein |
| At1g61180 | H3K4me3 + H3K36me2 | H3K4me2 | | Disease resistance protein |
| At1g62290 | H3K4me3 + H3K27me3 | | | Aspartyl protease family protein |
| At1g72450 | H3K4me3 + H3K36me2 | H3K4me2me3 | | [symbol] AZ6 |
| At2g04650 | H3K4me3 + H3K36me2 | | DNA b | Subunit RPE5b DNA-directed RNA polymerases |
| At2g15890 | H3K4me3 + H3K36me2 | H3K4me3 | | Expressed protein |
| At2g26600 | H3K4me3 + H3K36me2 | H3K4me1me2me3 | | Glycosyl hydrolase family 17 protein |
| At2g26860 | H3K4me3 + H3K36me2 | H3K4me2me3 | | F-box family protein |
| At2g28100 | H3K4me3 + H3K36me2 | H3K4me2me3 | | Glycosyl hydrolase family 29 |
| At2g37710 | H3K4me3 + H3K36me2 | H3K4me3 | | Lectin protein kinase |
| At2g40000 | H3K4me3 | H3K4me3 | | Expressed protein |
| At2g41100 | H3K4me3 + H3K27me3 + H3K36me2 | H3K4me2me3 | | TCH3, touch-responsive protein |
| At2g47060 | H3K4me3 + H3K36me2 | H3K4me2me3 | | Serine [symbol] theronine protein kinase |
| At3g04110 | H3K4me3 + H3K36me2 | H3K4me1me2me3 | | GLR1, glutamate receptor family protein |
| At3g06110 | H3K27me3 | H3K4me1me2me3 | | Dual specificity protein phosphatase family protein |
| At3g10500 | H3K4me3 + H3K36me2 | H3K4me1me2me3 | TF | ANAC053, no apical meris [symbol] (NAM) family protein |
| At3g15630 | H3K4me3 + H3K36me2 | H3K4me2me3 | | Expressed protein |
| At3g18000 | H3K4me3 + H3K36me2 | H3K4me1me2me3 | | NMT1, phosphoethanolamine N-methyltransferase 1 |
| At3g21220 | H3K4me3 | H3K4me2me3 | | AtMKK3, mitogen-activated protein kinase kinase |
| At3g24100 | H3K4me3 | H3K4me1me2me3 | | Four F5 family protein |
| At3g25740 | H3K4me3 | H3K4me3 | | MAP1B_MAP1C_{——}metallopeptidase |
| At3g30720 | H3K27me3 | H3K9me2 H3K4me2me3 | | Expressed protein |
| At3g48360 | H3K4me3 + H3K36me2 | H3K4me2me3 | TF | TAC1, TELOMERASE ACTIVATOR1 |
| At3g62220 | H3K4me3 + H3K27me3 + M3K36me2 | H3K4me2me3 | | Serine [symbol] threonine protein kinase |
| At4g01250 | H3K4me3 + H3K36me2 | H3K4me2me3 | TF | WRKY22_transcription factor |
| At4g03510 | H3K4me3 + H3K36me2 | H3K4me3 | | C3HC4-type RING finger family protein (RMA1) |
| At4g11640 | H3K4me3 + H3K27me3 + H3K36me2 | H3K4me2 | | Serine racemase |
| At4g25710 | H3K4me3 + H3K36me2 | H3K4me3 | | Kelch repeat-containing F-box family protein |
| At5g05040 | H3K27me3 | H3K27me3 | | Expressed protein |
| At5g05060 | H3K27me3 | H3K4me3 | | Expressed protein |
| At5g09570 | H3K27me3 | H3K4me1me2 | | Expressed protein |
| | | H3K27me3 | | |
| At5g10140 | H3K4me3 + H3K27me3 + H3K36me2 | H3K4me2me3 | TF | FLOWERING LOCUS C |
| At5g11250 | H3K27me3 | H3K4me1me2me3 | | Phosphate translocator-related |
| At5g22920 | H3K4me3 + H3K36me2 | H3K4me1me2me3 | | C3HC4-type RING finger family protein |
| At5g23405 | H3K4me3 + H3K36me2 | H3K4me3 | DNA b | High mobility group (HMG1/2) family protein |
| At5g37290 | H3K4me3 + H3K36me2 | H3K4me1me2me3 | | Armadillo [symbol] beta-catenin repeat family protein |
| At5g44870 | H3K4me3 + H3K27me3 + H3K36me2 | H3K4me1me2me3 | | Disease resistance protein (TIR-NBS-LRR class) |
| | | H3K27me3 | | |
| At5g46020 | H3K4me3 + H3K27me3 + M3K36me2 | H3K4me1me3 | | Expressed protein |
| At5g47230 | H3K4me3 + H3K36me2 | H3K4me2me3 | TF | ATERF5_(ethylene response factor) |
| At5g49450 | H3K4me3 + H3K36me2 | H3K4me2me3 | TF | bZIP family transcription factor |
| At5g50630 | H3K4me3 + H3K36me2 | | | Nodulin family protein |
| At5g56380 | H3K4me3 + H3K27me3 + HeK36me2 | H3K4me2me3 | | F-box family protein |
| | | H3K27me3 | | |
| At5g57220 | H3K27me3 | H3K4me2me3 | | CYP81F2, cytochrome P450 |
| At5g60680 | H3K4me3 | H3K4me3 H3K9me2 | | Expressed protein |
| At5g61590 | H3K4me3 + H3K36me2 | H3k4me2me3 | TF | ERF (ethylene responce factor) |

^{a}Bold, italics - downregulated in ashh2-1, sdg8-2 and ccr1; bold - ashh2-1 and ccr1; italics - sdg8-2 and ccr1; underlined - also downregulated in vip3.
^{b}According to http://epigenomics.mcdb.ucla.edu/.
^{c}Genes encoding transcription factors (TF) and DNA-binding (DNA b) proteins are indicated. [symbol]
indicates data missing or illegible when filed

TABLE IV

| | |
|---|---|
| Primer^{a} | Sequence^{b} |

Primers for real-time quantitative PCR

| | | |
|---|---|---|
| TUBS-FW | 5′ -ATAACCGTTTCAAATTCTCTCTCTC | SEQ ID NO: 31 |
| TUB8-RV | 5′ -TGCAAATCGTTCTCTCCTTG | SEQ ID NO: 32 |

Primers for ChIP (Chromatin immunoprecipitation

| | | |
|---|---|---|
| ACT2INT2_ANTISENSE | 5′ -CCGCAAGATCAAGACGAAGGATAGC | SEQ ID NO: 33 |
| ACT2INT2_SENSE | 5′ -CCCTGAGGAGCACCCAGTTCTACTC | SEQ ID NO: 34 |
| ANAC025 INTF | 5′ -GTTTCGGTTTCACCCGACTGATGAG | SEQ ID NO: 35 |
| ANAC025 INTR | 5′ -TCGCCTTGCCTAAACCATTCAAA | SEQ ID NO: 36 |
| AP1_PROBE_89_LP | 5′ -ATATGCCTCCCCCTCTGC | SEQ ID NO: 37 |
| AP1_PROBE_89_RP | 5′ -GGATCATCTTCTTGATACAGACCA | SEQ ID NO: 38 |
| ASP/ATDMC1 TSS52-251 | 5′ -ACCCACGTTTTCTCTCTTTCTC | SEQ ID NO: 39 |
| ASP/MYB99 TSS2 651-803 | 5′ -GGCCATTTTTTGTGATGCTTAA | SEQ ID NO: 40 |
| ATDMC1_PL_LP | 5′ -CAGGGTGTCAAGCTCTCGAT | SEQ ID NO: 41 |
| ATDMC1_PL_RP | 5′ -CTGTGATGGCTGAGGTTTCA | SEQ ID NO: 42 |
| FLC E V F2 | 5′ -CCCACAACACTTGTCTTCATGT | SEQ ID NO: 43 |
| FLC E V R2 | 5′ -TGACCAACATGGCCAAACTA | SEQ ID NO. 44 |
| FLC_C_V_F2 | 5′ -CGGTTCTGTGTTTGTTTGTGTT | SEQ ID NO: 45 |
| FLC_C_V_R2 | 5′ -TCGTGAATGACATGCAATTTT | SEQ ID NO: 46 |
| GAPA-38-L | 5′ -TCCATCGTCTCCTTCCAGAC | SEQ ID NO: 47 |
| GAPA-3S-R | 5′ -CTTGGCCTCAGTCACACCTT | SEQ ID NO: 48 |
| LTR.TA3-F | 5′ -TAGGGTTCTTAGTTGATCTTGTATTGAGCTC | SEQ ID NO: 49 |
| LTR.TA3-R | 5′ -TTTGCTCTCAAACTCTCAATTGAAGTTT | SEQ ID NO: 50 |
| MAF1 TSS2 ASP | 5′ -AGTGACTTGTCGACTGCTTT | SEQ ID NO: 51 |
| MAF1 TSS2 SP | 5′ -CCCTTATCGGAGATTTGAAG | SEQ ID NO: 52 |
| MAF1-D-F | 5′ -TCTCCAGCATTTCCAAGATC | SEQ ID NO: 53 |
| MAF1-D-R | 5′ -ACGGACAGAGCAGTCTCAAG | SEQ ID NO: 54 |
| MYB99 INTF | 5′ -CAAAACTGGCGGGGCTAAGGAG | SEQ ID NO: 55 |
| MYB99 INTR | 5′ -CTCCACTGCAATCTTCGACCATCTA | SEQ ID NO: 56 |
| SP/MYB99 TSS2 651-803 | 5′ -CACCGTATTCAATGGTTTTAGC | SEQ ID NO: 57 |
| SPATDMC1TSS 52-251 | 5′ -TGCTACGTAGATGAAACGAGTTT | SEQ ID NO: 58 |

Primers uses for site-directed mutagenesis of the ASHH2 CW domain.

| | | |
|---|---|---|
| ASHH2-CW-W874A FW | 5′ -TTGCTTTAAAGCGCGACGAATAC | SEQ ID NO: 59 |
| ASHH2-CW-W874A RV | 5′ -GTATTCGTCGCGCTTTAAAGCAA | SEQ ID NO: 60 |
| ASHH2-CW-W891A FW | 5′ -GAGCTCTAGAGCGATCTGTATGA | SEQ ID NO: 61 |
| ASHH2-CW-W891A RV | 5′ -TCATACAGATCGCTCTAGAGCTC | SEQ ID NO: 62 |
| ASHH2-CW-Q908A FW | 5′ -CTCAAAATCTGCAGAGATGTCAA | SEQ ID NO: 63 |
| ASHH2-CW-Q908A RV | 5′ -TTGACATCTCTGCAGATTTTGAG | SEQ ID NO: 64 |
| ASHH2-CW-E909A FW | 5′ -AAAATCTCAAGCGATGTCAAATG | SEQ ID NO: 65 |
| ASHH2-CW-E909A RV | 5′ -CATTTGACATCGCTTGAGATTTT | SEQ ID NO: 66 |

Primers for construction of the 07861.1 mutant

| | | |
|---|---|---|
| ASHH2-CW-W865A FW-mod | 5′ -cggaattcATGGTTGTGGATGTTA | SEQ ID NO: 67 |
| | CTATTGAAGATAGCTATTCCACA | |
| | GAGAGTGCCGCGGTTCGATGTG | |
| 3pGEX^{c} | 5′ -CCGGGAGCTGCATGTGTCAGAGG, | SEQ ID NO: 68 |

^{a}FW and ‘RV’ indicate forward and reverse primers, respectively.
^{b}Triplets in boldface indicate the codon mutated and the nucleotide changes are underlined.
^{c}A vector primer at the 3′-end.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

What is claimed is:

1. An isolated polypeptide comprising a CW domain operably linked to first member of a specific binding pair.

2. The isolated polypeptide of claim 1, wherein said CW domain is selected from the group consisting of Arabidopsis ASHH2, AtMDB1, AtMBD2, AtMBD3, AtMBD4, VAL1, VAL2, NP_{—}179516, FB304_ARATH, NP_{—}191849, O23424_ARATH CW domains and human and mouse ZCWPW1, ZCWPW2, MORC1, MORC2, MORC3 CW domains.

3. The isolated polypeptide of claim 2, wherein said CW domain is an Arabidopsis ASHH2 CW domain.

4. The isolated polypeptide of claim 1, wherein said first member of a specific binding pair is a protein tag.

5. The isolated polypeptide of claim 4, wherein said protein tag is selected from the group consisting of glutathione-S-transferase (GST), a His-tag, a maltose binding protein-tag, a SBP-tag, a Flag-tag, a HA-tag, and a Myc-tag.

6. A nucleic acid encoding the isolated polypeptide of claim 1.

7. An expression vector comprising the nucleic acid of claim 6.

8. A host cell comprising the nucleic acid of claim 6.

9. A system or kit for analysis of methylation of chromatin comprising:
a polypeptide comprising a CW domain operably linked to a first member of a specific binding pair; and
at least one reagent comprising a second member of said specific binding pair.

10. The system of claim 9, wherein said CW domain is a selected from the group consisting of Arabidopsis ASHH2, AtMDB1, AtMBD2, AtMBD3, AtMBD4, VAL1, VAL2, NP_{—}179516, FB304_ARATH, NP_{—}191849, O23424_ARATH CW domains and human and mouse ZCWPW1, ZCWPW2, MORC1, MORC2, MORC3 CW domains.

11. The system of claim 10, wherein said CW domain is an Arabidopsis ASHH2 CW domain.

12. The system of claim 9, wherein said first member of a specific binding pair is a protein tag.

13. The system of claim 12, wherein said protein tag is selected from the group consisting of glutathione-S-transferase (GST), a His-tag, a maltose binding protein-tag, a SBP-tag, a Flag-tag, a HA-tag, and a Myc-tag.

14. The system of claim 9, wherein said reagent comprising a second member of said specific binding pair comprises a media support.

15. The system of claim 9, wherein said media support is selected from the group consisting of magnetic beads, a polymeric beads, planar supports, and chromatography supports.

16. The system of claim 9, wherein said reagent comprising a second member of said specific binding pair comprises a member selected from the group consisting of glutathione, amylase, Ni, avidin, and an antibody specific for FLAG, HA or myc.

17. A method for analyzing methylation of chromatin comprising:
contacting a chromatin sample with a reagent comprising a CW domain polypeptide to form a reagent-chromatin complex; and
analyzing said reagent-chromatin complex.

18. The method of claim 17, wherein said analyzing comprises isolating said reagent-chromatin complex.

19. The method of claim 18, wherein said reagent further comprises a first member of a specific binding pair and said isolating further comprises contacting said complex with a reagent comprising a second member of said specific binding pair.

20. The method of claim 19, wherein said analyzing further comprises analysis of nucleic acid sequences associated with said chromatin.

21. An isolated polypeptide comprising a CW domain operably linked to an effector domain polypeptide.

22. The isolated polypeptide of claim 1, wherein said CW domain is selected from the group consisting of Arabidopsis ASHH2, AtMDB1, AtMBD2, AtMBD3, AtMBD4, VAL1, VAL2, NP_{—}179516, FB304_ARATH, NP_{—}191849, O23424_ARATH CW domains and human and mouse ZCWPW1, ZCWPW2, MORC1, MORC2, MORC3 CW domains.

23. The isolated polypeptide of claim 2, wherein said CW domain is an Arabidopsis ASHH2 CW domain.

24. The isolated polypeptide of claim 1, wherein said effector domain polypeptide reacts with DNA or nucleosomal histones.

25. A nucleic acid encoding the isolated polypeptide of claim 21.

26. An expression vector comprising the nucleic acid of claim 25.

27. A host cell comprising the nucleic acid of claim 25.

28. A transgenic organism comprising the vector of claim 26.

29. A method for altering the chromatin of a cell or organism comprising: introducing a vector according to claim 26 into a target cell or organism.
